(19) United States
(12) Patent Application Publication
(10) Pub. No.: US 2026/0273088 A1
(43) Pub. Date: Sep. 17, 2026

(54) PEGYLATED ORGANELLE COMPLEXES

(71) Applicant: LUCA Science Inc., Tokyo (JP)

(72) Inventors: Hideyoshi HARASHIMA, Sapporo (JP); Yuma YAMADA, Sapporo (JP); Masashi SUGANUMA, Toyota (JP); Rick C. TSAI, Tokyo (JP); Masahiro SHIRAISHI, Sapporo (JP)

(21) Appl. No.: 19/470,805

(22) PCT Filed: Mar. 29, 2024
(86) PCT No.: PCT/US2024/022284

§ 371 Date: Sep. 29, 2025

Related U.S. Application Data
(60) Provisional application No. 63/493,686, filed on Mar. 31, 2023.

(51) Int. Cl. A61K 47/69; A61K 45/06; A61K 47/54; A61K 47/60

(52) U.S. Cl. CPC A61K 47/6901; A61K 45/06; A61K 47/543; A61K 47/60

(57) ABSTRACT

According to the present invention, there is provided a composition comprising a population of mitochondria with a smaller size and a population of lipid membrane-based vesicles encapsulating mitochondria in a closed space and a method for producing the composition.

### CROSS REFERENCE TO RELATED APPLICATIONS

This application claims the benefit under 35 U.S.C. § 119(e) of U.S. Provisional Application No. 63/493,686 filed Mar. 31, 2023, which is hereby incorporated by reference herein in its entirety.

### BACKGROUND

### Field

The present disclosure relates generally to methods of generating coated organelle complexes, coated organelle complexes obtained by such methods, and uses of coated organelle complexes obtained by such methods.

### Description of the Related Art

Mitochondria are intracellular organelles responsible for a number of metabolic transformations and regulatory functions. They produce most of the ATP employed by eukaryotic cells. For mitochondrial function, the folded inner membrane and the surrounding outer membrane, and the electron transport system located in the inner membrane play a crucial role. The inner membrane forms a highly folded structure called cristae, which is believed to hold the supercomplex of electron transport system in the cristae membrane and to keep the proton concentration high by trapping the pumped protons in the cristae space. The electrochemical proton gradient formed by the electron transport system enables the transport of anions as well as ATP synthesis and cation transport.

Mitochondria are also highly dynamic organelles that move throughout the cell and undergo structural transitions, changing the length, morphology, shape, and size. Moreover, mitochondria are continuously eliminated and regenerated in a process known as mitochondrial biogenesis. While most mitochondrial genes have been transferred to the nuclear genome, the mitochondria genome still encodes rRNAs, tRNAs, and 13 subunits of the electron transport chain (ETC). Functional communication between the nuclear and mitochondrial genomes is therefore essential for mitochondrial biogenesis, efficient oxidative phosphorylation, and normal health. Mitochondria are also the major source of free radicals and reactive oxygen species (ROS) that cause oxidative stress. Additionally, mitochondria play key roles in intracellular signaling as well as control of cell death, including apoptosis and necrosis.

There is growing evidence that mitochondrial dysfunction is associated with a broad range of human diseases. Mitochondrial dysfunction, for example, respiratory chain complex dysfunction, is a major cause responsible for a mitochondrial disease and aging. Decreased mitochondrial function influences cells in many organs principally involved in mitochondrial diseases and age-related diseases. The introduction of exogenous mitochondria into cells of a subject in need is promising approach to treating or preventing a number of diseases and disorders. However, there is a need for population comprising mitochondria (e.g., organelle complexes) that possess enhanced stability, functionality, pharmacodynamic control, and targeting.

### SUMMARY

Disclosed herein include coated organelle complexes population. In some embodiments, the coated organelle complexes comprise mitochondria and one or more of endoplasmic reticulum, peroxisomes, lysosomes, and Golgi apparatus. In some embodiments, the surface of the coated organelle complexes comprises one or more lipid-polymer conjugates. In some embodiments, the one or more lipid-polymer conjugates comprise a polymer conjugated to a lipid. In some embodiments, the polymer is a bio-soluble polymer and/or a biodegradable polymer. In some embodiments, the one or more lipid-polymer conjugates are 1,2-dimyristoyl-sn-glycerol, methoxy polyethylene glycol 2000 (DMG-PEG 2000) and/or distearoyl-sn-glycero-3-phosphoethanolamine-N—, methoxy polyethylene glycol 2000 (DSPE-PEG 2000). In some embodiments, the one or more lipid-polymer conjugates does not comprise triphenylphosphonium (TPP), cholesterol, oleic acid, or any combination thereof.

In some embodiments, the coated organelle complexes comprise coated first organelle complexes, coated second organelle complexes, or a combination of coated first organelle complexes and coated second organelle complexes. In some embodiments, the coated first organelle complexes and coated second organelle complexes are depleted of cytosolic macromolecules. In some embodiments, coated first organelle complexes are derived from (i) frozen cells; (ii) floating cells; and/or (iii) cells contacted with a surfactant at a concentration at or above the critical micellar concentration (CMC) for the surfactant. In some embodiments, coated second organelle complexes are derived from (i) adherent cells; and/or (ii) cells contacted with a surfactant at a concentration below the critical micellar concentration (CMC) for the surfactant. In some embodiments, the cytosolic macromolecules comprise cytosolic proteins, wherein the abundance of one or more cytosolic proteins is depleted by at least about 90% as compared to the cells from which the organelle complexes population are derived. In some embodiments, the cytosolic proteins are p70S6K and/or glyceraldehyde 3-phosphate dehydrogenase (GAPDH).

In some embodiments, the coated organelle complexes comprise: one or more mitochondrial matrix proteins (e.g., mitochondrial transcription factor A (TFAM) and/or citrate synthase (CS)); one or more outer mitochondrial membrane proteins (e.g., outer mitochondrial membrane complex subunit 20 (TOMM20)); one or more lysosome proteins (e.g., lysosomal-associated membrane protein 2 (LAMP2), mannose-6-phosphate receptor (M6PR), and/or lysosomal-associated membrane protein 1 (LAMP1)); one or more peroxisome proteins (e.g., catalase and/or ATP-binding cassette transporter 1, subfamily D, type 3 (ABCD3)); one or more inner mitochondrial membrane proteins (e.g., respiratory chain proteins); mitochondrial DNA, mitochondrial RNA, or both; one or more Golgi apparatus proteins (e.g., Golgin-97, Sintaxin-6, TGOLN2/trans-Golgi network protein 2 (TGN46), Golgi matrix protein 130 (GM130), and/or Mannosidase Alpha Class 2A Member 1 (MAN2A1)); and/or one or more endoplasmic reticulum proteins (e.g., Calreticulin and/or Calnexin). In some embodiments, the organelle complexes are derived from cells treated with a mitochondria-activating agent (e.g., resveratrol).

In some embodiments, the lipid comprises an amphipathic lipid having a hydrophobic moiety and a hydrophilic portion. In some embodiments, the amphipathic lipid is selected from the group comprising phospholipids, aminolipids and sphingolipids. In some embodiments, the phospholipid is selected from the group comprising dimyristoylphosphatidylglycerol (DMG), distearoyl phosphatidyl-ethanolamine (DSPE), dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2 Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroylphosphatidylglycerol (DLPG), diarachidoylphosphatidylglycerol (DAPG), dipalmitoylphosphatidylglycerol (DPPG), distcaroylphosphatidylglycerol (DSPG), diolcoyl-phosphatidylglycerol (DOPG), dimyristoyl phosphatidic acid (DMPA), dipalmitoyl phosphatidic acid (DPPA), distearoyl phosphatidic acid (DSPA), diarachidoylphosphatidic acid (DAPA), dimyristoylphosphatidylethanolamine (DMPE), dipalmitoylphosphatidylethanolamine (DPPE), dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), and dioleoyl-phosphatidylinositol (DOPI). In some embodiments, the phospholipid comprises a saturated fatty acid with a C14-C20 carbon chain and/or an unsaturated fatty acid with a C14-C20 carbon chain. In some embodiments, the lipid comprises a phosphatidylethanolamine (e.g., phosphatidylethanolamine); has a carbon chain length of 10 to 20; comprises saturated fatty acids; comprises unsaturated fatty acids; comprises saturated fatty acids and unsaturated fatty acids; and/or is selected from the group comprising distearoylphosphatidyl-ethanolamine (DSPE), dimyristoylphosphatidylethanolamine (DMPE), dipalmitoylphosphatidylethanolamine (DPPE), and dioleoylphosphatidylethanolamine (DOPE).

In some embodiments, the polymer has a molecular weight: between about 100 and about 20000 daltons (Da); between about 100 and about 1000 Da; between about 1000 and about 3500 Da; between about 3500 and 7000 Da; and/or of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 Da. In some embodiments, the polymer is or comprises poly(acrylate), poly(methacrylate), poly(acrylic acid), poly(acrylamide), poly(vinylpyridine), poly(vinylpyrrolidone), poly(vinyl alcohol), a naturally-derived polymer, poly(ether), poly(maleic anhydride), poly(styrene sulfonate), poly(allylamine hydrochloride), poly(sulfone), poly(ethersulfone), poly(ethylene glycol), copolymers thereof, or any combination thereof. In some embodiments, the surface of the coated organelle complexes comprises the one or more lipid-polymer conjugates at a molar ratio of greater than at least about 0.1%, about 0.5%, about 1%, or about 5%, of the mass of the organelle complexes.

In some embodiments, the presence of the one or more lipid-polymer conjugate in the surface of the coated organelle complexes does not reduce mitochondrial function. In some embodiments, the presence of the one or more lipid-polymer conjugate in the surface of the coated organelle complexes reduces mitochondrial function less than about 10 percent, about 5 percent, or about 1 percent, as compared to organelle complexes not comprising the one or more lipid-polymer conjugates. In some embodiments, mitochondrial function comprises one or more of ATP production, outer membrane structural integrity of mitochondria, and cytochrome c oxidase (COX) activity.

In some embodiments, the polydispersity index (PDI) of the coated organelle complexes population is within about 5 percent, about 10 percent, about 15 percent, or about 20 percent, of the PDI of a population of organelle complexes not comprising the one or more lipid-polymer conjugates. In some embodiments, the ζ-potential of the coated organelle complexes population is at least about 5 percent, about 10 percent, about 25 percent, about 50 percent, about 75 percent, or about 100 percent, more positive as compared to the ζ-potential of a population of organelle complexes not comprising the one or more lipid-polymer conjugates. In some embodiments, the average diameter of the coated organelle complexes population is at least about 5 percent, about 10 percent, about 25 percent, about 50 percent, about 75 percent, or about 100 percent, smaller as compared to the average diameter of a population of organelle complexes not comprising the one or more lipid-polymer conjugates. In some embodiments, the stability of the coated organelle complexes population in solution is at least about 5 percent, about 10 percent, about 25 percent, about 50 percent, about 75 percent, or about 100 percent, greater than a population of organelle complexes not comprising the one or more lipid-polymer conjugates. In some embodiments, the physical stability of the coated organelle complexes population against internal stimuli and/or external stimuli is at least about 5 percent, about 10 percent, about 25 percent, about 50 percent, about 75 percent, or about 100 percent, greater as compared to a population of organelle complexes not comprising the one or more lipid-polymer conjugates. In some embodiments, at least about 70 percent, about 80 percent, about 90 percent, or about 100 percent, of the coated organelle complexes population is functional after the population undergoes one or more freeze-thaw cycles.

In some embodiments, one or both ends of the polymer is functionalized with a functional group selected from the group comprising vinyl, carboxylate, hydroxyl, epoxide, sulfhydryl, amide, acrylate, thiol, azide, maleimide, isocyanate, aziridine, carbonate, N-hydroxysuccinimide ester, imidoester, carbodiimide, anhydride, succinimidyl carbonate, and amine, or any combination thereof. In some embodiments, the one or more lipid-polymer conjugates further comprise a targeting agent (e.g., polyarginine). In some embodiments, upon contact of the coated organelle complexes population with a population of cells, the coated organelle complexes have at least about 1.1-fold superior incorporation capability into said cells as compared to organelle complexes not comprising the one or more lipid-polymer conjugates comprising the targeting agent. In some embodiments, the targeting agent is configured to bind a ligand on the surface of a target cell (e.g., a target cell of a subject in need). In some embodiments, the binding of the targeting agent and ligand causes the coated organelle complexes to be incorporated by the target cell. In some embodiments, the ligand is: differentially expressed between target cells and non-target cells; absent on non-target cells; and/or over expressed on target cells. In some embodiments, the target cells are residents of a target tissue, wherein said target tissue is cancerous, inflamed, damaged, dysfunctional, infected, the site of disease or disorder, and/or proximate to a site of a disease or disorder. In some embodiments, the tissue comprises adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. In some embodiments, the targeting agent is configured to bind axons. In some embodiments, the targeting agent is or comprises a peptide, an antigen binding domain, a cytokine, a chemokine, an aptamer, a growth factor, a hormone, a cytokine, an interleukin, a receptor, or any combination thereof. In some embodiments, the antigen binding domain comprises an antibody, an antibody fragment, an scFv, a Fv, a Fab, a (Fab′)2, a single domain antibody (SDAB), a VH or VL domain, a camelid VHH domain, a Fab′, a F(ab′)2, a Fv, a scFv, a dsFv, a diabody, a triabody, a tetrabody, a multispecific antibody formed from antibody fragments, a single-domain antibody (sdAb), a single chain comprising anticomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the one or more lipid-polymer conjugates further comprise a detectable moiety configured for detection of the coated organelle complexes in vivo and/or in vitro. In some embodiments, the detectable moiety comprises a fluorescent molecule (e.g., fluorescein amidite (FAM), fluorescein dyes, carbocyanine, merocyanine, styryl dyes, oxonol dyes, phycoerythrin, erythrosin, eosin, rhodamine dyes, coumarin, coumarin dyes, Oregon Green Dyes, Texas Red, Texas Red-X, Spectrum Red™, Spectrum Green™, cyanine dyes, Fluor dyes, BODIPY dyes, derivatives thereof, or any combination thereof). In some embodiments, the detectable moiety comprises a fluorescent protein (e.g., green fluorescent protein (GFP), enhanced GFP (EGFP), blue fluorescent proteins (BFP), cyan fluorescent protein (CFP), yellow fluorescent protein (YFP), red fluorescent protein (RFP), derivatives thereof, or any combination thereof). In some embodiments, the detectable moiety comprises a radioisotope that is detectable by Single Photon Emission Computed Tomography (SPECT) and/or Position Emission Tomography (PET). In some embodiments, the radioisotope is selected from the group comprising iodine-131 (¹³¹I), iodine-125 (¹²⁵I), bismuth-212 (²¹²Bi), bismuth-213 (²¹³Bi), astatine-211 (²¹¹At), copper-67 (⁶⁷Cu), copper-64 (⁶⁴Cu), rhenium-186 (¹⁸⁶Re), rhenium-188 (¹⁸⁸Re), phosphorus-32 (³²P), samarium-153 (¹⁵³Sm), lutetium-177 (¹⁷⁷Lu), technetium-99m (^{99m}Tc), gallium-67 (⁶⁷Ga), indium-111 (¹¹¹In), and thallium-201 (²⁰¹Tl). In some embodiments, the detectable moiety comprises a quantum dot (Qdot) fluorescent particle (e.g., Qdot525, Qdot565, Qdot585, Qdot605, Qdot625, Qdot655, Qdot705, Qdot800, derivatives thereof, or any combination thereof).

In some embodiments, the one or more lipid-polymer conjugates further comprise one or more secondary agents, such as, for example, a therapeutic agent (e.g., a small molecule drug). In some embodiments, one or more secondary agents is an anti-cancer agent, an anti-inflammatory agent, an anti-infective agent, a regenerative agent, a relaxing agent, an apoptosis-inhibiting agent, an apoptosis-inducing agent, an anti-coagulatory agent, an antioxidant molecule, an autophagy-inducing agent, a dermatological agent, a growth-stimulating agent, a vasodilating agent, a vasoconstricting agent, an analgesic agent, and an anti-allergic agent, condensate modifying drugs (c-MODS) or a combination thereof. In some embodiments, one or more secondary agents is a chemotherapeutic, a nucleic acid, a polysaccharide, a peptide, a polypeptide, or any combination thereof. In some embodiments, one or more secondary agents is a protein phosphatase inhibitor, a kinase inhibitor, a cytokine, an inhibitor of an immune inhibitory molecule, an immune modulator, an anti-metastatic, a chemotherapeutic, a hormone or a growth factor antagonist, an alkylating agent, a TLR agonist, a cytokine antagonist, a cytokine antagonist, or any combination thereof. In some embodiments, one or more secondary agents is an agonistic or antagonistic antibody specific to a checkpoint inhibitor or checkpoint stimulator molecule such as PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3.

Disclosed herein include methods for generating a coated organelle complexes population. In some embodiments, the method comprises: contacting organelle complexes in a first solution with one or more lipid-polymer conjugates to generate coated organelle complexes; and recovering coated organelle complexes from the first solution to generate the coated organelle complexes population. Also disclosed herein include coated organelle complexes population obtained by the methods provided herein. Also disclosed herein include populations of host cells comprising coated organelle complexes generated according to the methods provided herein.

In some embodiments, the method further comprises: incubating the first solution after the contacting step, such as, for example, for about 1 minute to about 120 minutes (e.g., about 15 minutes). In some embodiments, the contacting step comprises applying a physical stimulus to the first solution (e.g., shaking, mixing, pipetting, and/or stirring). In some embodiments, the incubating step is performed at a first temperature. In some embodiments, the organelle complexes are present in the first solution at concentration of about 0.01 mg/mL to about 10 mg/mL. In some embodiments, the organelle complexes are present in the first solution at concentration of about 0.1 mg/mL to about 1 mg/mL (e.g., about 1 mg/mL). In some embodiments, the contacting step comprises contacting the first solution with about 1 μL to about 1000 μL of a solution comprising the one or more lipid-polymer conjugates, and wherein the one or more lipid-polymer conjugates are present at a concentration of about 0.1 mM to about 10 mM (e.g., a 1 mM 100 μL solution). In some embodiments, recovering the coated organelle complexes from the first solution comprises one or more centrifugation steps. In some embodiments, recovering the coated organelle complexes from the first solution comprises: centrifuging the first solution at a first centrifugal force; collecting the pellet to recover the coated organelle complexes. In some embodiments, the first centrifugal force is about 100 g to about 10000 g (e.g., about 3000 g). In some embodiments, collecting the pellet comprises resuspending the coated organelle complexes population in a second solution. In some embodiments, the second solution has a volume of about 50 μL to about 50 mL. In some embodiments, the second solution has a volume of about 100 μL to about 1000 μL (e.g., about 1000 μL). In some embodiments, centrifuging step is performed for at least about 1 min, about 2 min, about 3 min, about 4 min, about 5 min, about 6 min, about 7 min, about 8 min, about 9 min, about 10 min, about 15 min, or about 20 min. In some embodiments, the centrifugation step is performed at a second temperature. In some embodiments, the first temperature and/or the second temperature is about 0° C. to about 50° C. In some embodiments, the first temperature is about 20° C. to about 25° C. and the second temperature is about 0° C. to about 4° C. In some embodiments, the recovering step depletes the one or more lipid-polymer conjugates that are not incorporated into the surface of the organelle complexes. In some embodiments, the one or more lipid-polymer conjugates that are not incorporated into the surface of the organelle complexes are present as micelles.

Disclosed herein include methods for introducing coated organelle complexes into host cells. In some embodiments, the method comprises: contacting a coated organelle complexes population disclosed herein with a population of host cells, wherein upon contact of the coated organelle complexes with the host cells, the coated organelle complexes are capable of incorporating into the host cells.

In some embodiments, the host cells comprise one or more mammalian cells selected from the group comprising an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof. In some embodiments, the host cells are the cell of a subject, such as, for example, a subject suffering from a disease or disorder. In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof. In some embodiments, the contacting is performed for a period time of at least about 1 hr, about 2 hr, about 3 hr, about 4 hr, about 5 hr, about 6 hr, about 7 hr, about 8 hr, about 9 hr, about 10 hr, about 12 hr, about 16 hr, about 20 hr, or about 24 hr.

In some embodiments, the method comprises attaching a targeting agent, a detectable moiety, and/or one or more secondary agents to the one or more lipid-polymer conjugates. In some embodiments, the polymer, the targeting agent, the detectable moiety, and/or the one or more secondary agents is functionalized with a functional group selected from the group comprising vinyl, carboxylate, hydroxyl, epoxide, sulfhydryl, amide, acrylate, thiol, azide, maleimide, isocyanate, aziridine, carbonate, N-hydroxysuccinimide ester, imidoester, carbodiimide, anhydride, succinimidyl carbonate, and amine, and combinations thereof. In some embodiments, the attaching comprises contacting the targeting agent, the detectable moiety, and/or the one or more secondary agents with the one or more lipid-polymer conjugates for at least about 1 min, about 2 min, about 3 min, about 4 min, about 5 min, about 6 min, about 7 min, about 8 min, about 9 min, about 10 min, about 15 min, about 20 min, about 30 min, about 40 min, about 50 min, or about 60 min. In some embodiments, the attaching is performed at a temperature of about 0° C. to about 50° C. (e.g., about 20° C. to about 25° C. or about 0° C. to about 4° C.). In some embodiments, upon contact of the coated organelle complexes population with the population of host cells, the coated organelle complexes have superior incorporation capability into host cells as compared to organelle complexes not comprising the one or more lipid-polymer conjugates. In some embodiments, said one or more lipid-polymer conjugates comprise a targeting agent configured to bind a ligand on the surface of said host cells.

Disclosed herein include compositions comprising: (i) a coated organelle complexes population disclosed herein, and/or (ii) a population of host cells comprising coated organelle complexes disclosed herein. Disclosed herein include pharmaceutical composition. In some embodiments, the pharmaceutical composition comprises: (i) a coated organelle complexes population disclosed herein, and/or (ii) a population of host cells comprising coated organelle complexes disclosed herein. In some embodiments, the pharmaceutical composition comprises: one or more pharmaceutically acceptable carriers and/or one or more secondary agents.

Disclosed herein include methods of treating or preventing a disease or disorder in a subject. In some embodiments, the method comprises contacting cells of a subject in need thereof with an effective amount of: (i) a coated organelle complexes population provided herein; (ii) a population of host cells comprising coated organelle complexes provided herein; (iii) a composition provided herein; and/or (iv) a pharmaceutical composition provided herein, thereby treating, or preventing the disease or disorder in the subject.

In some embodiments, the contacting is performed ex vivo, in vitro, or in vivo. In some embodiments, the effective amount comprises at least about 1 ug to about 1 mg of the coated organelle complexes population. In some embodiments, the subject is a mammal. In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the coated organelle complexes population is incorporated into target cell(s) and/or target tissue(s) of the subject. In some embodiments, less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the coated organelle complexes population are incorporated into non-target cell(s) and/or non-target tissue(s) of the subject.

In some embodiments, the disease or disorder is selected from the group consisting of diabetes (Type I and Type II), metabolic disease, ocular disorders associated with mitochondrial dysfunction, hearing loss, mitochondrial toxicity associated with therapeutic agents, mitochondrial dysfunction associated with Space travel, cardiotoxicity associated with chemotherapy or other therapeutic agents, a mitochondrial dysfunction disorder, and migraine. In some embodiments, the disease or disorder is selected from the group consisting of mitochondrial myopathy, diabetes and deafness (DAD) syndrome, Barth Syndrome, Leber's hereditary optic neuropathy (LHON), Leigh syndrome, NARP (neuropathy, ataxia, retinitis pigmentosa and ptosis syndrome), myoneurogenic gastrointestinal encephalopathy (MNGIE), MELAS (mitochondrial encephalopathy, lactic acidosis, and stroke-like episodes) syndrome, myoclonic epilepsy with ragged red fibers (MERRF) syndrome, Kearns-Sayre syndrome, and mitochondrial DNA depletion syndrome. In some embodiments, the disease or disorder is an ischemia-related disease or disorder, a genetic disorder, an aging disease or disorder, a neurodegenerative condition, a cardiovascular condition, a cancer, an autoimmune disease, an inflammatory disease, a fibrotic disorder, or any combination thereof. In some embodiments, the ischemia-related disease or disorder is selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, critical limb ischemia and reperfusion injury of a transplanted organ. In some embodiments, the neurodegenerative condition is selected from the group consisting of dementia, Friedrich's ataxia, amyotrophic lateral sclerosis, mitochondrial encephalopathy, lactic acidosis, and stroke-like episodes (MELAS), myoclonic epilepsy with ragged red fibers (MERFF), epilepsy, Parkinson's disease, Alzheimer's disease, or Huntington's Disease. Exemplary neuropsychiatric disorders include bipolar disorder, schizophrenia, depression, addiction disorders, anxiety disorders, attention deficit disorders, personality disorders, autism, and Asperger's disease. In some embodiments, the cardiovascular condition is selected from the group consisting of coronary heart disease, myocardial infarction, atherosclerosis, high blood pressure, cardiac arrest, cerebrovascular disease, peripheral arterial disease, rheumatic heart disease, congenital heart disease, congestive heart failure, arrhythmia, stroke, deep vein thrombosis, and pulmonary embolism. In some embodiments, the disease or disorder is acute respiratory distress syndrome (ARDS) or pre-eclampsia or intrauterine growth restriction (IUGR) or fetal growth restriction (FGR).

In some embodiments, the disease or disorder is associated with expression of a tumor antigen, and wherein the disease associated with expression of a tumor antigen is selected from the group consisting of a proliferative disease, a precancerous condition, a cancer, and a non-cancer related indication associated with expression of the tumor antigen. In some embodiments, the cancer is selected from the group consisting of colon cancer, rectal cancer, renal-cell carcinoma, liver cancer, small cell or non-small cell carcinoma of the lung, mesothelioma, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, the contacting comprises systemic administration, intrathecal administration, intracranial injection, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, inhalation, intrapulmonary administration, and intra-ocular administration, or any combination thereof. In some embodiments, the systemic administration is intravenous, intramuscular, intraperitoneal, or intraarticular.

### BRIEF DESCRIPTION OF THE DRAWINGS

FIG. 1 depicts a non-limiting exemplary schematic of a coated mitochondrion provided herein.

FIGS. 2A-2D depict data related to size distributions of isolated mitochondria alone (PEG(−); FIG. 2A), DMG-PEG 1 mM (FIG. 2B), DMG-PEG 3 mM (FIG. 2C), and DMG-PEG 5 mM (FIG. 2D).

FIGS. 3A-3D depict data related to size distributions of isolated mitochondria alone (PEG(−); FIG. 3A), DSPE-PEG 1 mM (FIG. 3B), DSPE-PEG 3 mM (FIG. 3C), and DSPE-PEG 5 mM (FIG. 3D).

FIGS. 4A-4B depict data related to size distribution (FIG. 4A), zeta potential distribution (FIG. 4B).

FIGS. 5A-5C depict data related to diameter (FIG. 5A), PdI (FIG. 5B), and ζ-potential (FIG. 5C) before and after the centrifugation step. *: p<0.05 by two-tail unpaired t test (Mean±S.D. n=3).

FIGS. 6A-6C depict data related to diameter (FIG. 6A), PdI (FIG. 6B), and ζ-potential (FIG. 6C) before and after PEG modification. *: p<0.05, **: p<0.01 by two-tail unpaired t test (Mean±S.D. n=3).

FIG. 7 depicts data related to PEG content of control second organelle complexes (Q+tris) and coated second organelle complexes (Q+PEG). **: p<0.01 by two-tail unpaired t test. Mean±S.D. (n=3).

FIGS. 8A-8B depict data related to ATP level (FIG. 8A) and outer membrane integrity (FIG. 8B) for intact (untreated second organelle complexes (Q)), PEG coated second organelle complexes (PEG-Q), and control second organelle complexes (Tris-Q). nrANOVA followed by SNK test. n.s. (Mean±S.D. n=3).

FIGS. 9A-9B depict non-limiting exemplary schematics showing a PEGylated mitochondrion wherein the PEG-lipid conjugate is peptide modified (FIG. 9A) and a maleimide (mal) group attached to PEG (FIG. 9B).

FIGS. 10A-10B depict data related to unmodified second organelle complexes (Q) reacted in presence/absence of peptide (FIG. 10A) and coated second organelle complexes (PEG (mal)-Q) reacted at the indicated PEG: peptide molar ratios (FIG. 10B) at 25° C. for 1 hr.

FIGS. 11A-11B depict data related to peptide modification of second organelle complexes at 4° C. FIG. 11A depicts coated second organelle complexes (PEG (mal)-Q) reacted at indicated PEG: peptide molar ratios. FIG. 11B depicts coated second organelle complexes (PEG (mal)-Q) reacted with peptide for the indicated time period or without peptide.

FIGS. 12A-12C depict data related to depict data related to diameter (FIG. 12A), PdI (FIG. 12B), and ζ-potential (FIG. 12C) of PEG(mal)-Q (DMG-PEG 2000 maleimide+Q), Pep PEG(mal)-Q (Peptide+DMG-PEG 2000 maleimide+Q), Pep PEG-Q (Peptide+DMG-PEG 2000+Q), and Pep-Q (Peptide+Q). nrANOVA followed by SNK test. *: p<0.05, **: p<0.01. Mean±S.D (n=3-9).

FIGS. 13A-13D depict FACS data related to PEG(mal)-Q (DMG-PEG 2000 maleimide+Q) versus Pep PEG(mal)-Q (Peptide+DMG-PEG 2000 maleimide+Q) (FIG. 13A), PEG(mal)-Q versus Pep PEG-Q (Peptide+DMG-PEG 2000+Q) (FIG. 13B), PEG(mal)-Q versus Pep-Q (Peptide+Q) (FIG. 13C), and all four modification conditions (FIG. 13D). nrANOVA followed by SNK test. *: p<0.05, **: p<0.01. Mean±S.D (n=3-9).

FIGS. 14A-14G depict a schematic (FIG. 14A) and data (FIGS. 14B-14G) related to uptake of Peptide+PEG-modified HEKQ by HEK cells. nrANOVA followed by SNK test. FIG. 14B depicts FACS data related to Peptide(−) versus Peptide(+) modification conditions with HEKQ/RFP. FIG. 14C depicts FACS data related to pepPEG-HEKQRFP (PEG+peptide HEKQ/RFP), PEG-HEKQRFP (PEG HEKQ/RFP), HEKQRFP (HEKQ/RFP) and non-treated (NT). FIGS. 14D-14E depict FITC-A (FAM) data and FIGS. 14F-14G depict PE-A (RFP) FACS data. *: p<0.05, **: p<0.01. Mean±S.D (n=3).

FIGS. 15A-15C depict data related to the impact of incubation time on uptake of HEKQ (FIG. 15A), PEG-HEKQ (FIG. 15B), and PepPEG-HEKQ (FIG. 15C). nrANOVA followed by SNK test. **: p<0.01. Mean±S.D (n=3).

FIGS. 16A-16D depict data related to the mitochondrial function of peptide-modified PEG-Q with regards to ATP production (FIGS. 16A-16B), outer membrane integrity (FIG. 16C), and Cytochrome C Oxidase activity (FIG. 16D).

FIG. 17 depict data related to the effect of administration time on uptake of R8-PEG-HEKQ.

FIGS. 18A-18C depict data related to the respiratory function of peptide-modified PEG-Q.

FIGS. 19A-19C depict data related to the respiratory function of peptide-modified PEG-Q.

FIGS. 20A-20B depict data related to the respiratory function of peptide-modified PEG-Q.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Unless defined otherwise, technical, and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, “isolated” shall be given its ordinary meaning and shall also refer to a substance or entity that has been (1) separated from at least some of the components with which it was associated when initially produced (whether in nature or in an experimental setting), and/or (2) produced, prepared, and/or manufactured by the hand of man. In some embodiments, an isolated mitochondrion or isolated organelle complexes population has been processed to obtain it from a cellular environment via the methods provided herein.

As used herein, the term “mitochondrion” shall be given its ordinary meaning and shall also refer to an organelle present in a eukaryotic cell that has double-layered lipid membranes, the inner and outer membranes, and a matrix surrounded by cristae and inner membranes. Mitochondria (more than one mitochondrion) have enzymes on their inner membrane, such as the respiratory chain complexes, which is involved in oxidative phosphorylation. The inner membrane has a membrane potential due to the internal-external proton gradients formed by the action of the respiratory chain complexes, etc. Mitochondria are thought to be unable to maintain the membrane potential when the inner membrane is disrupted. As used herein, the term “organelle complex” shall be given its ordinary meaning and shall also refer to a complex of mitochondria and one or more of endoplasmic reticulum, peroxisomes, lysosomes, and Golgi apparatus. As used herein, the term “coated organelle complex” shall be given its ordinary meaning and shall also refer to an organelle complex having a surface comprising one or more of the lipid-polymer conjugates provided herein. Organelle complexes can be depleted of cytosolic macromolecules (e.g., cytosolic proteins). In some embodiments, organelle complexes do not comprise cytosolic macromolecules. In some embodiments, an organelle complexes population comprises homogenized mitochondria. As used herein, the term “population” shall be given its ordinary meaning and shall also refer to a group of a plurality of the same or different substances. For example, an “organelle complexes population” is a group of at least a plurality of the same or different organelle complexes. The population may not be always homogenous and may have physical, chemical and/or physiological distributions. The physical distribution includes, for example, particle size and polydispersity index. The chemical distribution includes, for example, a zeta potential distribution and a lipid composition distribution. The physiological distribution includes, for example, a difference of physiological function (for example, respiratory activity). An organelle complexes population can comprise first organelle complexes, second organelle complexes, homogenized mitochondria, or any combination thereof. As used herein, the term “homogenized mitochondria” shall be given its ordinary meaning and shall also refer to mitochondria isolated via a method comprising one or more homogenization steps.

As used herein, the term “surfactant” shall be given its ordinary meaning and shall also refer to a molecule having a hydrophilic moiety and a hydrophobic moiety in one molecule. Surfactants have the role of reducing surface tension at the interface or mixing polar and non-polar substances by forming micelles. Surfactants are roughly classified into nonionic surfactants and ionic surfactants. Nonionic surfactants are those in which the hydrophilic moiety is not ionized, and ionic surfactants are those in which the hydrophilic moiety comprises either a cation or an anion or both a cation and an anion. As used herein, the term “critical micelle concentration” (CMC) shall be given its ordinary meaning and shall also refer to the concentration at which, when the concentration is reached, the surfactant forms micelles, and the surfactant further added to the system contributes to micelle formation, in particular the concentration in bulk. At concentrations above the critical micelle concentration, the addition of surfactants to the system ideally increases the amount of micelles, especially the number of micelles.

As used herein, a “subject” refers to an animal that is the object of treatment, observation, or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports, and pet animals. Non-limiting examples of mammals include mice; rats; rabbits; guinea pigs; dogs; cats; sheep; goats; cows; horses; primates, such as monkeys, chimpanzees, and apes, and, in particular, humans. In some embodiments, the mammal is a human. However, in some embodiments, the mammal is not a human. As used herein, the term “host cell” shall be given its ordinary meaning and shall also refer to an in vivo cell, an in vitro cell, and/or an ex vivo cell into which the incorporation of exogenous mitochondria and/or coated organelle complexes is intended.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder, or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method. As used herein, the term “effective amount” refers to an amount sufficient to effect beneficial or desirable biological and/or clinical results.

As used herein, the term “contacting” shall be given its ordinary meaning and shall also refer to placing two or more entities in such proximity such that they actually physically contact each other, e.g., by combining the two or more entities (e.g., coated organelle complexes and host cells). Contacting can comprise co-incubation. Contacting can occur in vitro, in situ or in vivo. In some embodiments, contacting the two entities comprises incorporation (e.g., transplantation) of one entity into another entity physically contacted. Contacting coated organelle complexes with a population of host cells can comprise contacting a coated organelle complexes population with a population of host cells. Contacting coated organelle complexes with a population of host cells can generate a population of host cells comprising exogenous organelle complexes. Upon contact of the coated organelle complexes with a population of host cells, the coated organelle complexes provided herein can be capable of incorporating into the host cells. In some embodiments, incorporation of coated organelle complexes (e.g., transplantation) into a host cell comprises colocalization and/or fusion with endogenous mitochondria within said host cell. The host cells can be in vivo, in vitro, or ex vivo. In some embodiments, coated organelle complexes that have been incorporated (e.g., transplanted) into host cells can be detected (e.g., distinguished from the endogenous organelles of the host cells) for at least a period of time (e.g., 6 hours, 12 hours, 16 hours, 1 day, 2 days, 3 days, 4 days, 5 days, 6 days, 7 days, or a number or a range between any two of these values). The beneficial effects of coated organelle complexes transplantation provided herein can persist beyond the time when said coated organelle complexes are detectable within a host cell population.

Disclosed herein include coated organelle complexes population. In some embodiments, the coated organelle complexes comprise mitochondria and one or more of endoplasmic reticulum, peroxisomes, lysosomes, and Golgi apparatus. In some embodiments, the surface of the coated organelle complexes comprises one or more lipid-polymer conjugates. In some embodiments, the one or more lipid-polymer conjugates comprise a polymer conjugated to a lipid. In some embodiments, the polymer is a bio-soluble polymer and/or a biodegradable polymer. In some embodiments, the one or more lipid-polymer conjugates are 1,2-dimyristoyl-sn-glycerol, methoxy polyethylene glycol 2000 (DMG-PEG 2000) and/or distearoyl-sn-glycero-3-phosphoethanolamine-N—, methoxy polyethylene glycol 2000 (DSPE-PEG 2000). In some embodiments, the one or more lipid-polymer conjugates does not comprise triphenylphosphonium (TPP), cholesterol, oleic acid, or any combination thereof.

Disclosed herein include methods for generating a coated organelle complexes population. In some embodiments, the method comprises: contacting organelle complexes in a first solution with one or more lipid-polymer conjugates to generate coated organelle complexes; and recovering coated organelle complexes from the first solution to generate the coated organelle complexes population. Also disclosed herein include coated organelle complexes population obtained by the methods provided herein. Also disclosed herein include population of host cells comprising coated organelle complexes generated according to the methods provided herein.

Disclosed herein include methods for introducing coated organelle complexes into host cells. In some embodiments, the method comprises: contacting a coated organelle complexes population disclosed herein with a population of host cells, wherein upon contact of the coated organelle complexes with the host cells, the coated organelle complexes are capable of incorporating into the host cells.

Disclosed herein include compositions comprising: (i) a coated organelle complexes population disclosed herein, and/or (ii) a population of host cells comprising coated organelle complexes disclosed herein. Disclosed herein include pharmaceutical composition. In some embodiments, the pharmaceutical composition comprises: (i) a coated organelle complexes population disclosed herein, and/or (ii) a population of host cells comprising coated organelle complexes disclosed herein. In some embodiments, the pharmaceutical composition comprises: one or more pharmaceutically acceptable carriers and/or one or more secondary agents.

Disclosed herein include methods of treating or preventing a disease or disorder in a subject. In some embodiments, the method comprises contacting cells of a subject in need thereof with an effective amount of: (i) a coated organelle complexes population provided herein; (ii) a population of host cells comprising coated organelle complexes provided herein; (iii) a composition provided herein; and/or (iv) a pharmaceutical composition provided herein, thereby treating or preventing the disease or disorder in the subject.

The methods, compositions, systems, and kits provided herein can, in some embodiments, be employed in concert with the methods, compositions, systems, and kits described in PCT Patent Application Publication Nos. WO2018/092839, WO2017/090763, WO2020/230601, WO2019/164003, WO2020/054824, WO2020/203961, WO2020/054829, WO2021/015298, and WO2021/132735, the contents of which are incorporated herein by reference in their entirety. The methods, compositions, systems, and kits provided herein can, in some embodiments, be employed in concert with the methods, compositions, systems, and kits described in U.S. Patent Application No. 63/359,108, entitled, “REDOX-MODULATING ORGANELLE COMPLEXES,” filed Jul. 7, 2022 and PCT Patent Application Publication No. WO2024/010866, and in U.S. Patent Application No. 63/406,022, entitled, “METHODS OF IMPROVING CELLULAR THERAPY WITH ORGANELLE COMPLEXES,” filed Sep. 13, 2022 and PCT Patent Application Publication No. WO2024/030441, the contents of which are incorporated herein by reference in their entirety.

### Coated Organelle Complexes

Disclosed herein include coated organelle complexes population. The coated organelle complexes can comprise mitochondria and one or more of endoplasmic reticulum, peroxisomes, lysosomes, and Golgi apparatus. The surface of the coated organelle complexes can comprise one or more lipid-polymer conjugates. The one or more lipid-polymer conjugates can comprise a polymer conjugated to a lipid. The polymer can be a bio-soluble polymer and/or a biodegradable polymer. The one or more lipid-polymer conjugates can be 1,2-dimyristoyl-sn-glycerol, methoxy polyethylene glycol 2000 (DMG-PEG 2000) and/or distearoyl-sn-glycero-3-phosphoethanolamine-N—, methoxy polyethylene glycol 2000 (DSPE-PEG 2000). In some embodiments, the one or more lipid-polymer conjugates does not comprise triphenylphosphonium (TPP), cholesterol, oleic acid, or any combination thereof. One or both ends of the polymer can be functionalized with a functional group selected from the group comprising vinyl, carboxylate, hydroxyl, epoxide, sulfhydryl, amide, acrylate, thiol, azide, maleimide, isocyanate, aziridine, carbonate, N-hydroxysuccinimide ester, imidoester, carbodiimide, anhydride, succinimidyl carbonate, and amine, or any combination thereof. Also disclosed herein include coated organelle complexes population obtained by the methods provided herein.

The lipid can comprise an amphipathic lipid having a hydrophobic moiety and a hydrophilic portion. The amphipathic lipid can be selected from the group comprising phospholipids, aminolipids and sphingolipids. The phospholipid can be selected from the group comprising dimyristoylphosphatidylglycerol (DMG), distearoyl phosphatidyl-ethanolamine (DSPE), dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2 Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroylphosphatidylglycerol (DLPG), diarachidoylphosphatidylglycerol (DAPG), dipalmitoylphosphatidylglycerol (DPPG), distearoylphosphatidylglycerol (DSPG), dioleoyl-phosphatidylglycerol (DOPG), dimyristoyl phosphatidic acid (DMPA), dipalmitoyl phosphatidic acid (DPPA), distearoyl phosphatidic acid (DSPA), diarachidoylphosphatidic acid (DAPA), dimyristoylphosphatidylethanolamine (DMPE), dipalmitoylphosphatidylethanolamine (DPPE), dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distcaroylphosphatidylinositol (DSPI), and diolcoyl-phosphatidylinositol (DOPI). The phospholipid can comprise a saturated fatty acid with a C14-C20 carbon chain and/or an unsaturated fatty acid with a C14-C20 carbon chain. The lipid can comprise a phosphatidylethanolamine (e.g., phosphatidylethanolamine). The lipid can have a carbon chain length of 10 to 20. The lipid can comprise saturated fatty acids. The lipid can comprise unsaturated fatty acids. The lipid can comprise saturated fatty acids and unsaturated fatty acids. The lipid can be selected from the group comprising distearoylphosphatidyl-ethanolamine (DSPE), dimyristoylphosphatidylethanolamine (DMPE), dipalmitoylphosphatidylethanolamine (DPPE), and dioleoylphosphatidylethanolamine (DOPE).

The polymer can have a molecular weight: between about 100 and about 20000 daltons (Da); between about 100 and about 1000 Da; between about 1000 and about 3500 Da; between about 3500 and 7000 Da; and/or of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000 Da, or a number or a range between any two of these values. The polymer can be or can comprise poly(acrylate), poly(methacrylate), poly(acrylic acid), poly(acrylamide), poly(vinylpyridine), poly(vinylpyrrolidone), poly(vinyl alcohol), a naturally-derived polymer, poly(ether), poly(maleic anhydride), poly(styrene sulfonate), poly(allylamine hydrochloride), poly(sulfone), poly(ethersulfone), poly(ethylene glycol), copolymers thereof, or any combination thereof. The surface of the coated organelle complexes can comprise the one or more lipid-polymer conjugates at a molar ratio of greater than at least about 0.1%, about 0.5%, about 1%, about 2%, about 3%, about 4%, about 5%, or a number or a range between any two of these values, of the mass of the organelle complexes.

In some embodiments, the presence of the one or more lipid-polymer conjugate in the surface of the coated organelle complexes does not reduce mitochondrial function. In some embodiments, the presence of the one or more lipid-polymer conjugate in the surface of the coated organelle complexes reduces mitochondrial function less than about 10 percent, about 5 percent, or about 1 percent, or a number or a range between any two of these values, as compared to organelle complexes not comprising the one or more lipid-polymer conjugates. Mitochondrial function can comprise one or more of ATP production, outer membrane structural integrity of mitochondria, and cytochrome c oxidase (COX) activity.

The polydispersity index (PDI) of the coated organelle complexes population can be within about 5%, about 10%, about 15%, about 20%, or a number or a range between any two of these values, of the PDI of a population of organelle complexes not comprising the one or more lipid-polymer conjugates. The ζ-potential of the coated organelle complexes population can be at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, more positive as compared to the ζ-potential of a population of organelle complexes not comprising the one or more lipid-polymer conjugates. The average diameter of the coated organelle complexes population can be at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, smaller as compared to the average diameter of a population of organelle complexes not comprising the one or more lipid-polymer conjugates.

The stability of the coated organelle complexes population in solution can be at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, greater than a population of organelle complexes not comprising the one or more lipid-polymer conjugates. The physical stability of the coated organelle complexes population against internal stimuli and/or external stimuli can be at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, greater as compared to a population of organelle complexes not comprising the one or more lipid-polymer conjugates. In some embodiments, at least about 70%, about 75%, about 80%, about 85%, about 90%, about 95%, about 100%, or a number or a range between any two of these values, of the coated organelle complexes population can be functional after the population undergoes one or more freeze-thaw cycles.

The coated organelle complexes provided herein can comprise first organelle complexes, coated second organelle complexes, or a combination of coated first organelle complexes and coated second organelle complexes. The coated organelle complexes (e.g., coated first organelle complexes, coated second organelle complexes) provided herein can comprise mitochondria and one, two, three, or four of endoplasmic reticulum, peroxisomes, lysosomes, and Golgi apparatus. The coated first organelle complexes and coated second organelle complexes can be depleted of cytosolic macromolecules. Coated first organelle complexes can be derived from (i) frozen cells; (ii) floating cells; and/or (iii) cells contacted with a surfactant at a concentration at or above the critical micellar concentration (CMC) for the surfactant. Coated second organelle complexes can be derived from (i) adherent cells; and/or (ii) cells contacted with a surfactant at a concentration below the critical micellar concentration (CMC) for the surfactant. The cytosolic macromolecules can comprise cytosolic proteins, and the abundance of one or more cytosolic proteins can be depleted by at least about 90% as compared to the cells from which the organelle complexes population are derived. The cytosolic proteins can be p70S6K and/or glyceraldehyde 3-phosphate dehydrogenase (GAPDH). The coated organelle complexes can comprise: one or more mitochondrial matrix proteins (e.g., mitochondrial transcription factor A (TFAM) and/or citrate synthase (CS)); one or more outer mitochondrial membrane proteins (e.g., outer mitochondrial membrane complex subunit 20 (TOMM20)); one or more lysosome proteins (e.g., lysosomal-associated membrane protein 2 (LAMP2), mannose-6-phosphate receptor (M6PR), and/or lysosomal-associated membrane protein 1 (LAMP1)); one or more peroxisome proteins (e.g., catalase and/or ATP-binding cassette transporter 1, subfamily D, type 3 (ABCD3)); one or more inner mitochondrial membrane proteins (e.g., respiratory chain proteins); mitochondrial DNA, mitochondrial RNA, or both; one or more Golgi apparatus proteins (e.g., Golgin-97, Sintaxin-6, TGOLN2/trans-Golgi network protein 2 (TGN46), Golgi matrix protein 130 (GM130), and/or Mannosidase Alpha Class 2A Member 1 (MAN2A1)); and/or one or more endoplasmic reticulum proteins (e.g., Calreticulin and/or Calnexin).

The coated organelle complexes (e.g., coated first organelle complexes, coated second organelle complexes) can comprise: (i) mitochondria and endoplasmic reticulum; (ii) mitochondria and peroxisomes; (iii) mitochondria and lysosomes; (iv) mitochondria and Golgi apparatus; (v) mitochondria, endoplasmic reticulum, and peroxisomes; (vi) mitochondria, endoplasmic reticulum, and lysosomes; (vii) mitochondria, endoplasmic reticulum, and Golgi apparatus; (viii) mitochondria, endoplasmic reticulum, peroxisomes, and lysosomes; (ix) mitochondria, endoplasmic reticulum, peroxisomes, and Golgi apparatus; (x) mitochondria, endoplasmic reticulum, peroxisomes, lysosomes, and Golgi apparatus; (xi) mitochondria, endoplasmic reticulum, lysosomes, and Golgi apparatus; (xii) mitochondria, peroxisomes, and lysosomes; (xiii) mitochondria, peroxisomes, and Golgi apparatus; (xiv) mitochondria, peroxisomes, lysosomes, and Golgi apparatus; and/or (xv) mitochondria, lysosomes, and Golgi apparatus. The ratio of mitochondria to additional organelles (e.g., endoplasmic reticulum, peroxisomes, lysosomes, and/or Golgi apparatus) in the coated organelle complexes population can vary.

Provided herein are coated first organelle complexes generated from first organelle complexes populations using the methods provided herein. Also provided herein are coated second organelle complexes generated from second organelle complexes populations using the methods provided herein. Disclosed herein include methods for generating first organelle complexes populations. In some embodiments, the method comprises: incubating cells in a first solution comprising a surfactant at a first temperature; removing the surfactant to form a second solution; and recovering first organelle complexes from the second solution. First organelle complexes can be derived from: (i) frozen cells; (ii) floating cells; and/or (iii) cells contacted with a surfactant at a concentration at or above the critical micellar concentration (CMC) for the surfactant. The generation of first organelle complexes can comprise: (Step A) providing adherent, floating, and/or frozen cells, thawing, and placing in a tube. The generation of first organelle complexes can comprise: (Step A) providing adherent, floating, and/or frozen cells, centrifuging, and collecting the precipitant. The generation of first organelle complexes can comprise: (Step A) providing adherent cells, aspirating, adding a solution (e.g., PBS(−)), aspirating, adding TrypLE, incubating, adding a solution (e.g., PBS(−)), placing the cell suspension in a tube, centrifuging, and collecting the precipitant. The generation of first organelle complexes can comprise one or more of the following steps: (Step B) adding Tris Buffer, centrifuging, and collecting the precipitant; (Step C) adding Tris Buffer and vortexing; (Step D) adding a solution comprising a surfactant and incubating; (Step E) centrifuging and collecting the precipitant; (Step F) adding Tris Buffer, centrifuging and collecting the precipitant; (Step G) adding Tris Buffer and pipetting; (Step H) transferring to another tube and collecting buffer solution in the original tube and rinsing it; (Step I) centrifuging and collecting the supernatant; (Step J) centrifuging and collecting the precipitant; and (Step K) pipetting. One or more of the above steps can comprise an incubation period. One or more of the above steps can comprise a centrifugation step, followed by collection of the supernatant and/or the precipitant. One or more of the above steps can be omitted and one or more additional steps can be included. The times, volumes, concentrations, and centrifugal forces can vary depending on the embodiment. Methods of obtaining organelle complexes from cells and organelle complexes obtained by such methods are disclosed in U.S. Patent Application No. 63/359,110, entitled “ORGANELLE COMPLEXES”, filed on Jul. 7, 2022 and PCT Patent Application Publication No. WO2024/010862, the contents of which are incorporated herein by reference in its entirety. There are provided, in some embodiments, second organelle complexes. In some embodiments, the method for isolating second organelle complexes from cells comprises treating cells in a first solution with a surfactant at a concentration below the critical micelle concentration (CMC) for the surfactant, removing the surfactant to form a second solution, incubating the cells in the second solution, and recovering second organelle complexes from the second solution. Second organelle complexes can be derived from: (i) adherent cells; and/or (ii) cells contacted with a surfactant at a concentration below the critical micellar concentration (CMC) for the surfactant. There are provided in some embodiments, Q mitochondria. Second organelle complexes can comprise or be Q mitochondria. Methods of obtaining Q mitochondria from cells and Q mitochondria obtained by such methods are disclosed in PCT Patent Application Publication No. WO/2021/015298, the contents of which are incorporated herein by reference in its entirety. The organelle complexes population can be derived from cells treated with a mitochondria-activating agent (e.g., resveratrol). The organelle complexes can be depleted of cytosolic macromolecules. Cytosolic macromolecules can be absent from the organelle complexes populations provided herein. Organelle complexes (e.g., first organelle complexes, second organelle complexes) populations provided herein can comprise a negligible and/or undetectable amount of cytosolic macromolecules. The cytosolic macromolecules can comprise cytosolic proteins (e.g., p70S6K and/or glyceraldehyde 3-phosphate dehydrogenase (GAPDH)). The first organelle complexes and second organelle complexes can be derived from cells treated with a mitochondria-activating agent. The homogenized mitochondria, first organelle complexes, and/or second organelle complexes can be encapsulated in lipid membrane-based vesicles. Methods of encapsulating in lipid membrane-based vesicles are disclosed in PCT Patent Application Publication No. WO2021/132735, the contents of which are incorporated herein by reference in its entirety.

### Targeting Agents

In some embodiments, the one or more lipid-polymer conjugates further comprise a targeting agent (e.g., polyarginine). In some embodiments, upon contact of the coated organelle complexes population with a population of cells, the coated organelle complexes have at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) superior incorporation capability into said cells as compared to organelle complexes not comprising the one or more lipid-polymer conjugates comprising the targeting agent. In some embodiments, the targeting agent can be configured to bind a ligand on the surface of a target cell (e.g., a target cell of a subject in need). The binding of the targeting agent and ligand can cause the coated organelle complexes to be incorporated by the target cell.

In some embodiments, the ligand is: differentially expressed between target cells and non-target cells; absent on non-target cells; and/or over expressed on target cells. The target cells can be residents of a target tissue. Said target tissue can be cancerous, inflamed, damaged, dysfunctional, infected, the site of disease or disorder, and/or proximate to a site of a disease or disorder. The tissue can comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. The targeting agent can be configured to bind axons.

The targeting agent can be or can comprise a peptide, an antigen binding domain, a cytokine, a chemokine, an aptamer, a growth factor, a hormone, a cytokine, an interleukin, a receptor, or any combination thereof. The antigen binding domain can comprise an antibody, an antibody fragment, an scFv, a Fv, a Fab, a (Fab′)2, a single domain antibody (SDAB), a VH or VL domain, a camelid VHH domain, a Fab′, a F(ab′)2, a Fv, a scFv, a dsFv, a diabody, a triabody, a tetrabody, a multispecific antibody formed from antibody fragments, a single-domain antibody (sdAb), a single chain comprising anticomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

### Detectable Moieties

In some embodiments, the one or more lipid-polymer conjugates further comprise a detectable moiety configured for detection of the coated organelle complexes in vivo and/or in vitro. The detectable moiety can comprise a fluorescent molecule (e.g., fluorescein amidite (FAM), fluorescein dyes, carbocyanine, merocyanine, styryl dyes, oxonol dyes, phycoerythrin, erythrosin, eosin, rhodamine dyes, coumarin, coumarin dyes, Oregon Green Dyes, Texas Red, Texas Red-X, Spectrum Red™, Spectrum Green™, cyanine dyes, Fluor dyes, BODIPY dyes, derivatives thereof, or any combination thereof). The detectable moiety can comprise a fluorescent protein (e.g., green fluorescent protein (GFP), enhanced GFP (EGFP), blue fluorescent proteins (BFP), cyan fluorescent protein (CFP), yellow fluorescent protein (YFP), red fluorescent protein (RFP), derivatives thereof, or any combination thereof). The detectable moiety can comprise a radioisotope that is detectable by Single Photon Emission Computed Tomography (SPECT) and/or Position Emission Tomography (PET). The radioisotope can be selected from the group comprising iodine-131 (¹³¹I), iodine-125 (¹²⁵I), bismuth-212 (²¹²Bi), bismuth-213 (²¹³Bi), astatine-211 (²¹¹At), copper-67 (⁶⁷Cu), copper-64 (⁶⁴Cu), rhenium-186 (¹⁸⁶Re), rhenium-188 (¹⁸⁸Re), phosphorus-32 (³²P), samarium-153 (¹⁵³Sm), lutetium-177 (¹⁷⁷Lu), technetium-99m (^{99m}Tc), gallium-67 (⁶⁷Ga), indium-111 (¹¹¹In), and thallium-201 (²⁰¹Tl). The detectable moiety can comprise a quantum dot (Qdot) fluorescent particle (e.g., Qdot525, Qdot565, Qdot585, Qdot605, Qdot625, Qdot655, Qdot705, Qdot800, derivatives thereof, or any combination thereof).

### Secondary Agents

In some embodiments, the one or more lipid-polymer conjugates further comprise one or more secondary agents, such as, for example, a therapeutic agent (e.g., a small molecule drug). The one or more secondary agents can be an anti-cancer agent, an anti-inflammatory agent, an anti-infective agent, a regenerative agent, a relaxing agent, an apoptosis-inhibiting agent, an apoptosis-inducing agent, an anti-coagulatory agent, an antioxidant molecule, an autophagy-inducing agent, a dermatological agent, a growth-stimulating agent, a vasodilating agent, a vasoconstricting agent, an analgesic agent, and an anti-allergic agent, condensate modifying drugs (c-MODS) or a combination thereof. The one or more secondary agents can be a chemotherapeutic, a nucleic acid, a polysaccharide, a peptide, a polypeptide, or any combination thereof. The one or more secondary agents can be a protein phosphatase inhibitor, a kinase inhibitor, a cytokine, an inhibitor of an immune inhibitory molecule, an immune modulator, an anti-metastatic, a chemotherapeutic, a hormone or a growth factor antagonist, an alkylating agent, a TLR agonist, a cytokine antagonist, a cytokine antagonist, or any combination thereof. The one or more secondary agents can be an agonistic or antagonistic antibody specific to a checkpoint inhibitor or checkpoint stimulator molecule such as PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3.

### Methods of Generating Coated Organelle Complexes

Disclosed herein include methods for generating a coated organelle complexes population. In some embodiments, the method comprises: contacting organelle complexes in a first solution with one or more lipid-polymer conjugates to generate coated organelle complexes; and recovering coated organelle complexes from the first solution to generate the coated organelle complexes population. In some embodiments, the method further comprises: incubating the first solution after the contacting step, such as, for example, for about 1 minute to about 120 minutes (e.g., about 15 minutes). The contacting step can comprise applying a physical stimulus to the first solution (e.g., shaking, mixing, pipetting, and/or stirring). The incubating step can be performed at a first temperature. The organelle complexes can be present in the first solution at concentration of about 0.01 mg/mL to about 10 mg/mL. The organelle complexes can be present in the first solution at concentration of about 0.1 mg/mL to about 1 mg/mL (e.g., about 1 mg/mL). The amount of organelle complexes in the first solution can be, can be about, can be at least, or can be at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, ug, mg, ug/mL, and/or mg/mL.

The contacting step can comprise contacting the first solution with about 1 μL to about 1000 μL of a solution comprising the one or more lipid-polymer conjugates, and the one or more lipid-polymer conjugates can be present at a concentration of about 0.1 mM to about 10 mM (e.g., a 1 mM 100 μL solution). The one or more lipid-polymer conjugates can be present at a concentration of about 0.1, 0.2, 0.3, 0.4, 0.5, 0.6, 0.7, 0.8, 0.9, 1, 2, 3, 4, 5, 6, 7, 8, 9, 10, or a number or a range between any two of these values, mM. Recovering the coated organelle complexes from the first solution can comprise one or more centrifugation steps. In some embodiments, recovering the coated organelle complexes from the first solution comprises: centrifuging the first solution at a first centrifugal force; collecting the pellet to recover the coated organelle complexes. The first centrifugal force can be about 100 g to about 10000 g (e.g., about 3000 g). The first centrifugal force can be, can be about, can be at least, or can be at most, 100 g, 110 g, 120 g, 128 g, 130 g, 140 g, 150 g, 160 g, 170 g, 180 g, 190 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 510 g, 520 g, 530 g, 540 g, 550 g, 560 g, 570 g, 580 g, 590 g, 600 g, 610 g, 620 g, 630 g, 640 g, 650 g, 660 g, 670 g, 680 g, 690 g, 700 g, 710 g, 720 g, 730 g, 740 g, 750 g, 760 g, 770 g, 780 g, 790 g, 800 g, 810 g, 820 g, 830 g, 840 g, 850 g, 860 g, 870 g, 880 g, 890 g, 900 g, 910 g, 920 g, 930 g, 940 g, 950 g, 960 g, 970 g, 980 g, 990 g, 1000 g, 1100 g, 1200 g, 1300 g, 1400 g, 1500 g, 1600 g, 1700 g, 1800 g, 1900 g, 2000 g, 2100 g, 2200 g, 2300 g, 2400 g, 2500 g, 2600 g, 2700 g, 2800 g, 2900 g, 3000 g, 3250 g, 3500 g, 3750 g, 4000 g, 4250 g, 4500 g, 4750 g, 5000 g, 5500 g, 6000 g, 6500 g, 7000 g, 7500 g, 8000 g, 8500 g, 9000 g, 9500 g, 10000 g, or a number or a range between any two of these values.

Collecting the pellet can comprise resuspending the coated organelle complexes population in a second solution. The second solution can have a volume of about 50 μL to about 50 mL. The second solution can have a volume of about 100 μL to about 1000 μL (e.g., about 1000 μL). The first solution, the solution comprising the one or more lipid-polymer conjugates, and/or the second solution can be, can be about, can be at least, or can be at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, μL, and/or mL.

The centrifuging step can be performed for at least about 1 min, about 2 min, about 3 min, about 4 min, about 5 min, about 6 min, about 7 min, about 8 min, about 9 min, about 10 min, about 15 min, about 20 min, or a number or a range between any two of these values. The centrifugation step can be performed at a second temperature. The first temperature and/or the second temperature can be about 0° C. to about 50° C. The first temperature can be about 20° C. to about 25° C. and the second temperature can be about 0° C. to about 4° C. The first temperature and/or second temperature can be, can be about, can be at least, or can be at most, 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., or a number or a range between any two of these values. In some embodiments, the recovering step depletes the one or more lipid-polymer conjugates that are not incorporated into the surface of the organelle complexes. In some embodiments, the one or more lipid-polymer conjugates that are not incorporated into the surface of the organelle complexes are present as micelles.

Methods of Introducing Coated Organelle Complexes into Host Cells

Disclosed herein include methods for introducing coated organelle complexes into host cells. In some embodiments, the method comprises: contacting a coated organelle complexes population disclosed herein with a population of host cells, wherein upon contact of the coated organelle complexes with the host cells, the coated organelle complexes are capable of incorporating into the host cells. Also disclosed herein include population of host cells comprising coated organelle complexes generated according to the methods provided herein. The host cells can comprise one or more mammalian cells selected from the group comprising an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

The host cells can be the cell of a subject, such as, for example, a subject suffering from a disease or disorder. The disease or disorder can be a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof. The contacting can be performed for a period time of about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values, minutes.

The method can comprise attaching a targeting agent, a detectable moiety, and/or one or more secondary agents to the one or more lipid-polymer conjugates. The polymer, the targeting agent, the detectable moiety, and/or the one or more secondary agents can be functionalized with a functional group selected from the group comprising vinyl, carboxylate, hydroxyl, epoxide, sulfhydryl, amide, acrylate, thiol, azide, maleimide, isocyanate, aziridine, carbonate, N-hydroxysuccinimide ester, imidoester, carbodiimide, anhydride, succinimidyl carbonate, and amine, and combinations thereof. The attaching can comprise contacting the targeting agent, the detectable moiety, and/or the one or more secondary agents with the one or more lipid-polymer conjugates for at least about 1 min, about 2 min, about 3 min, about 4 min, about 5 min, about 6 min, about 7 min, about 8 min, about 9 min, about 10 min, about 15 min, about 20 min, about 30 min, about 40 min, about 50 min, about 60 min, or a number or a range between any two of these values. The attaching can be performed at a temperature of about 0° C. to about 50° C. (e.g., about 20° C. to about 25° C. or about 0° C. to about 4° C.). The temperature at which the attaching is performed can be, can be about, can be at least, or can be at most, 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., or a number or a range between any two of these values.

In some embodiments, upon contact of the coated organelle complexes population with the population of host cells, the coated organelle complexes have superior incorporation capability into host cells as compared to organelle complexes not comprising the one or more lipid-polymer conjugates. The one or more lipid-polymer conjugates can comprise a targeting agent configured to bind a ligand on the surface of said host cells.

### Methods of Treatment

There are provided, in some embodiments provided herein, methods of treating or preventing a disease or disorder in a subject. In some embodiments, the method comprises contacting cells of a subject in need thereof with an effective amount of: (i) a coated organelle complexes population provided herein; (ii) a population of host cells comprising coated organelle complexes provided herein; (iii) a composition provided herein; and/or (iv) a pharmaceutical composition provided herein, thereby treating or preventing the disease or disorder in the subject. The contacting can be performed ex vivo, in vitro, or in vivo. The effective amount of coated organelle complexes can be, can be about, can be at least, or can be at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, ug, mg, ug/mL, and/or mg/mL.

The subject can be a mammal. At least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, of the coated organelle complexes population can be incorporated into target cell(s) and/or target tissue(s) of the subject. Less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, of the coated organelle complexes population can be incorporated into non-target cell(s) and/or non-target tissue(s) of the subject.

The disease or disorder can be selected from the group consisting of diabetes (Type I and Type II), metabolic disease, ocular disorders associated with mitochondrial dysfunction, hearing loss, mitochondrial toxicity associated with therapeutic agents, mitochondrial dysfunction associated with Space travel, cardiotoxicity associated with chemotherapy or other therapeutic agents, a mitochondrial dysfunction disorder, and migraine.

The disease or disorder can be selected from the group consisting of mitochondrial myopathy, diabetes and deafness (DAD) syndrome, Barth Syndrome, Leber's hereditary optic neuropathy (LHON), Leigh syndrome, NARP (neuropathy, ataxia, retinitis pigmentosa and ptosis syndrome), myoneurogenic gastrointestinal encephalopathy (MNGIE), MELAS (mitochondrial encephalopathy, lactic acidosis, and stroke-like episodes) syndrome, myoclonic epilepsy with ragged red fibers (MERRF) syndrome, Kearns-Sayre syndrome, and mitochondrial DNA depletion syndrome.

The disease or disorder can be an ischemia-related disease or disorder, a genetic disorder, an aging disease or disorder, a neurodegenerative condition, a cardiovascular condition, a cancer, an autoimmune disease, an inflammatory disease, a fibrotic disorder, or any combination thereof. The ischemia-related disease or disorder can be selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, critical limb ischemia and reperfusion injury of a transplanted organ. The neurodegenerative condition can be selected from the group consisting of dementia, Friedrich's ataxia, amyotrophic lateral sclerosis, mitochondrial encephalopathy, lactic acidosis, and stroke-like episodes (MELAS), myoclonic epilepsy with ragged red fibers (MERFF), epilepsy, Parkinson's disease, Alzheimer's disease, or Huntington's Disease. Exemplary neuropsychiatric disorders include bipolar disorder, schizophrenia, depression, addiction disorders, anxiety disorders, attention deficit disorders, personality disorders, autism, and Asperger's disease. The cardiovascular condition can be selected from the group consisting of coronary heart disease, myocardial infarction, atherosclerosis, high blood pressure, cardiac arrest, cerebrovascular disease, peripheral arterial disease, rheumatic heart disease, congenital heart disease, congestive heart failure, arrhythmia, stroke, deep vein thrombosis, and pulmonary embolism. The disease or disorder can be acute respiratory distress syndrome (ARDS) or pre-eclampsia or intrauterine growth restriction (IUGR) or fetal growth restriction (FGR).

The disease or disorder can be associated with expression of a tumor antigen, and the disease associated with expression of a tumor antigen can be selected from the group consisting of a proliferative disease, a precancerous condition, a cancer, and a non-cancer related indication associated with expression of the tumor antigen.

The cancer can be selected from the group consisting of colon cancer, rectal cancer, renal-cell carcinoma, liver cancer, small cell or non-small cell carcinoma of the lung, mesothelioma, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers, combinations of said cancers, and metastatic lesions of said cancers.

The cancer can be a hematologic cancer chosen from one or more of chronic lymphocytic leukemia (CLL), acute leukemias, acute lymphoid leukemia (ALL), B-cell acute lymphoid leukemia (B-ALL), T cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

### Pharmaceutically Acceptable Compositions and Methods of Administration

There are provided, in some embodiments, pharmaceutical compositions. In some embodiments, the pharmaceutical composition comprises: (i) a coated organelle complexes population disclosed herein, and/or (ii) a population of host cells comprising coated organelle complexes disclosed herein. The pharmaceutical composition can comprise one or more pharmaceutically acceptable carriers and/or one or more secondary agents. Disclosed herein include compositions comprising: (i) a coated organelle complexes population disclosed herein, and/or (ii) a population of host cells comprising coated organelle complexes disclosed herein.

The present disclosure also provides use of coated organelle complexes in the manufacture of a medicament for treating the diseases and disorders provided herein. In some embodiments, the coated organelle complexes are administered to the subject in combination with one or more additional agents and/or additional therapies designed to treat the disease or disorder.

Contacting cells of the subject can comprise a route of administration selected from the group comprising intravenous administration, intra-arterial administration, intra-tracheal administration, subcutaneous administration, intramuscular administration, inhalation, intrapulmonary administration, and intra-ocular administration. The population of coated organelle complexes can be administered locally or systemically.

The wording “local administration” or “topic administration” as used herein indicates any route of administration by which a population of coated organelle complexes is brought in contact with the body of the individual, so that the resulting coated organelle complexes location in the body is topic (limited to a specific tissue, organ, or other body part where the imaging is desired). Exemplary local administration routes include injection into a particular tissue by a needle, gavage into the gastrointestinal tract, and spreading a solution containing a population of coated organelle complexes on a skin surface.

The wording “systemic administration” as used herein indicates any route of administration by which coated organelle complexes are brought in contact with the body of the individual, so that the resulting coated organelle complexes location in the body is systemic (i.e., non limited to a specific tissue, organ, or other body part where the imaging is desired). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

In another aspect, this disclosure provides pharmaceutically acceptable compositions which comprise a therapeutically-effective amount of coated organelle complexes disclosed herein. As described in detail below, the pharmaceutical compositions of this disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the coated organelle complexes. The pharmaceutical compositions can comprise one or more pharmaceutically-acceptable carriers. The phrase “therapeutically-effective amount” as used herein can refer to that amount of coated organelle complexes disclosed herein which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, vehicle, excipient, solvent, or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of this disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient (e.g., a population of coated organelle complexes) which can be combined with a carrier material to produce a single dosage form will vary depending upon the host being treated, the particular mode of administration. The amount of active ingredient, which can be combined with a carrier material to produce a single dosage form will generally be that amount of the coated organelle complexes which produces a therapeutic effect. Generally, out of one hundred percent, this amount will range from about 1% to about 99% of active ingredient, preferably from about 5% to about 70%, most preferably from about 10% to about 30%.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of coated organelle complexes include powders, sprays, ointments, pastes, creams, lotions, gels, solutions, patches, and inhalants. The active component may be mixed under sterile conditions with a pharmaceutically-acceptable carrier, and with any preservatives, buffers, or propellants which may be required.

The ointments, pastes, creams, and gels may contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Ophthalmic formulations, eye ointments, powders, solutions, and the like, are also contemplated as being within the scope of this disclosure.

Examples of suitable aqueous and nonaqueous carriers which may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Actual dosage levels of the active ingredients in the pharmaceutical compositions of this disclosure may be determined by the methods of this disclosure so as to obtain an amount of the active ingredient, which is effective to achieve the desired therapeutic response for a particular subject, composition, and mode of administration, without being toxic to the subject.

Also provided herein are kits comprising one or more compositions (e.g., a formulation comprising a population of coated organelle complexes) described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein.

### EXAMPLES

Some aspects of the embodiments discussed above are disclosed in further detail in the following examples, which are not in any way intended to limit the scope of the present disclosure.

### Example 1

### Coated Organelle Complexes

### Strategy

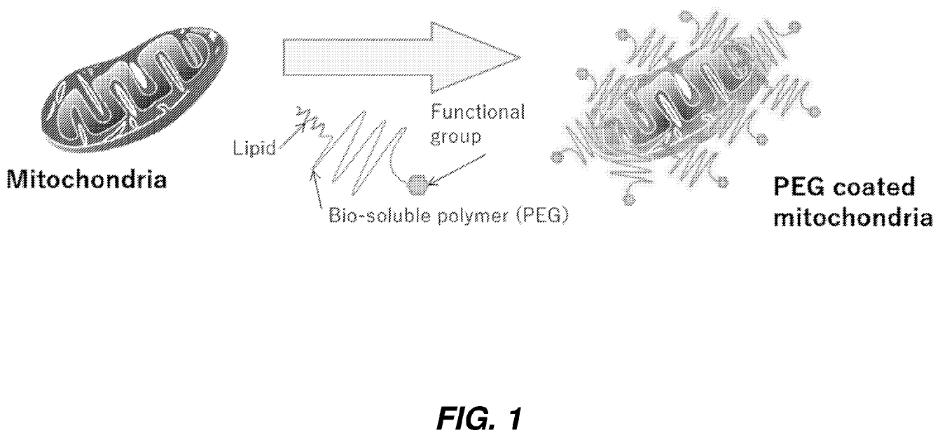

In order to increase the selectivity of organelle complexes to organs/cells, as well as to promote the stability of isolated organelle complexes, coating to the outer membrane of mitochondria were evaluated in this Example. For example, in some embodiments, lipid conjugated with bio-soluble polymer (PEG) is inserted into the outer membrane of mitochondria. FIG. 1 depicts a non-limiting exemplary schematic of a coated mitochondrion provided herein. Coating with the lipid-polymer conjugates disclosed herein can increase physical stability against external stimuli. In some embodiments, and without being bound by any particular theory, use of the lipid-polymer conjugates provided herein can improve stability of mitochondria in solution by increasing dispersion. Further, coating of the membrane of mitochondria with specific ligands such as specific tissue, cell surface and cell penetration oriented molecules can lead to pharmacodynamic control of mitochondria in some embodiments.

Various methods for insertion of lipid-polymer conjugates (e.g., PEG-lipid) into the outer membrane of mitochondria were investigated. An approach tested was pipetting (a gentle mixing method) and can comprise gentle mixing of mitochondria with excess amount of PEG-conjugated lipid for 15 min at room temperature. With this approach, there can be passive insertion of the lipid part of PEG-conjugated lipids into the outer membrane of mitochondria (e.g., an approach similar to liposome (lipid nano particle) modification). In this Example, use of DMG-PEG2000 (C14) and DSPE-2000 (C18) were evaluated. 1,2-dimyristoyl-sn-glycerol, methoxy polyethylene glycol 2000 (DMG-PEG 2000) has as a lipid anchor DMG, which has 2 anchor, carbon chain 14, and no double bonds. distearoyl-sn-glycero-3-phosphoethanolamine-N—, methoxy polyethylene glycol 2000 (DSPE-PEG 2000) has as a lipid anchor DSPE, which has 2 anchor, carbon chain 18, and no double bonds. Measurements were performed with regards to diameter for maintenance of original mitochondria size and zeta potential for insertion of PEG-lipid into mitochondria.

### Evaluation of DMG-PEG

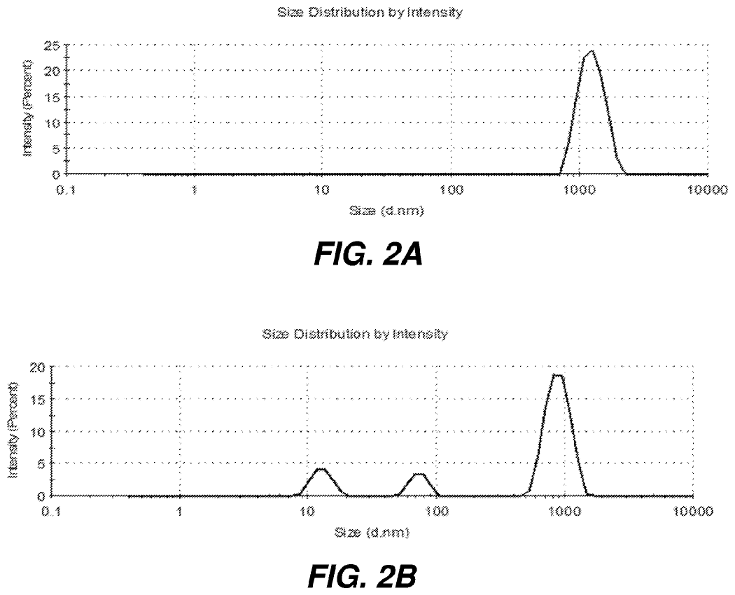

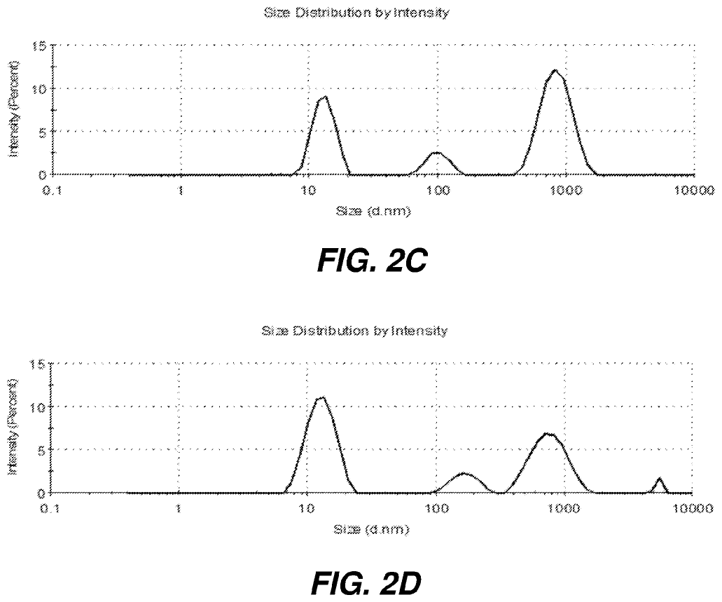

A pipetting-based gentle mixing method was employed. Mitochondria were gently mixed with an excess amount of PEG conjugated lipid for 15 min at room temperature. 1~5 mM of DMG-PEG (25 μL) was placed into a tube comprising 0.1 mg/mL mitochondria (25 μL). Following pipetting 3 times with a wide-mouth tip, a 15 min room temperature incubation was conducted for PEG-Lipid anchoring. Tables 1-2 and FIGS. 2A-2D depict data related to size distributions of isolated mitochondria alone (PEG(−); FIG. 2A), DMG-PEG 1 mM (FIG. 2B), DMG-PEG 3 mM (FIG. 2C), and DMG-PEG 5 mM (FIG. 2D). There is a peak at around 10 nm, which might be due to micelles of PEG-lipid. There was reduction of zeta potential due to coating of PEG with mitochondria. There were small particles detected, which was hypothesized to be PEG-lipid micelles.

TABLE 1
Isolated Mitochondria Alone (PEG(−))

| | Diameter | | ζ-Potential |
|---|---|---|---|
| | (nm) | PdI | (mV) |
| 2.5 μg | 1119 | 0.18 | −31.4 |

TABLE 2
Mitochondria and DMG-PEG (1 mM, 3 mM, 5 mM)

| | Diameter | | ζ-Potential |
|---|---|---|---|
| DMG-PEG | (nm) | PdI | (mV) |
| 1 mM | 511.3 | 0.72 | −20.0 |
| 3 mM | 233.6 | 0.62 | −14.4 |
| 5 mM | 127.1 | 0.48 | −15.2 |

### Evaluation of DSPE-PEG

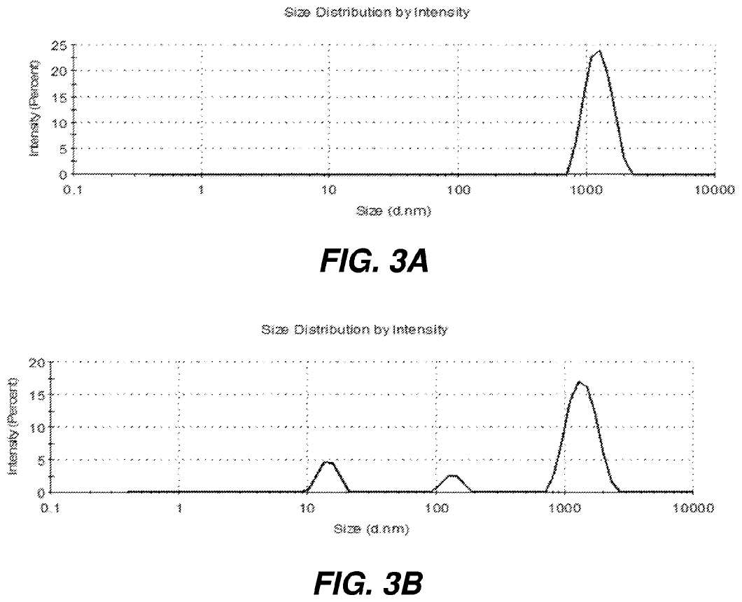

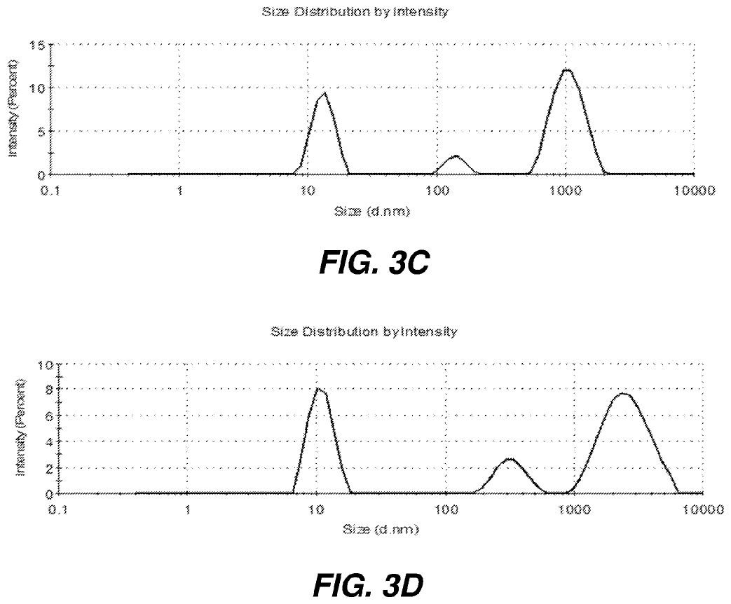

A pipetting-based gentle mixing method was employed. Mitochondria were gently mixed with an excess amount of PEG conjugated lipid for 15 min at room temperature. 1~5 mM DSPE-PEG (25 μL) was added to a tube comprising 0.1 mg/mL mitochondria (25 μL). Following pipetting 3 times with a wide-mouth tip, a 15 min room temperature incubation was conducted for PEG-Lipid anchoring. Tables 3-4 and FIGS. 3A-3D depict data related to size distributions of isolated mitochondria alone (PEG(−); FIG. 3A), DSPE-PEG 1 mM (FIG. 3B), DSPE-PEG 3 mM (FIG. 3C), and DSPE-PEG 5 mM (FIG. 3D). There is a peak at around 10 nm, which might be due to micelles of PEG-lipid. There was a slight reduction in zeta potential. There were small particles detected, which was hypothesized to be PEG-lipid micelles.

TABLE 3
Isolated Mitochondria Alone (PEG(−))

| | Diameter | | ζ-Potential |
|---|---|---|---|
| | (nm) | PdI | (mV) |
| 2.5 μg | 1119 | 0.18 | −31.4 |

TABLE 4
Mitochondria and DSPE-PEG (1 mM, 3 mM, 5 mM)

| | Diameter | | ζ-Potential |
|---|---|---|---|
| DSPE-PEG | (nm) | PdI | (mV) |
| 1 mM | 293.1 | 1.00 | −26.5 |
| 3 mM | 238.2 | 0.79 | −28.5 |
| 5 mM | 104.7 | 0.91 | −24.7 |

### Evaluation of Purification Process

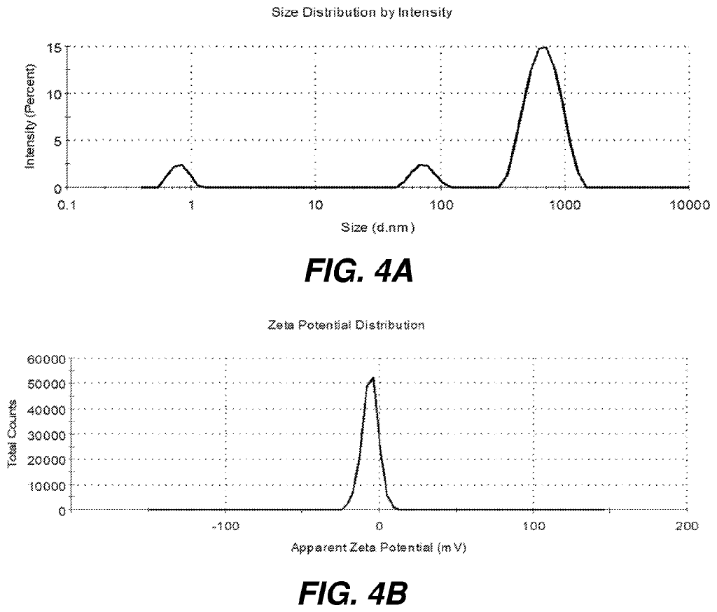

A purification process to remove excess PEG-lipid (micelle) was evaluated. 1 mM DMG-PEG (100 μL) was added to a tube having 0.1 mg/mL mitochondria (100 μL). After pipetting 3 times with a wide-mouth tip, a 15 min room temperature incubation was conducted for PEG-Lipid anchoring. Following the incubation, centrifugation was performed at 4° C. for 10 min at 10,000 g for removing micelles. Following centrifugation, the pellet comprises PEG coated mitochondria and the supernatant comprises PEG-lipid micelles. The supernatant was removed and the pellet was resuspended with Tris buffer (100 μL). FIGS. 4A-4B depict data related to size distribution (FIG. 4A), zeta potential distribution (FIG. 4B). A diameter of 375 nm and a ζ-Potential of −5.8 mV were observed. Zeta potential shifted toward neutral due to anchoring PEG.

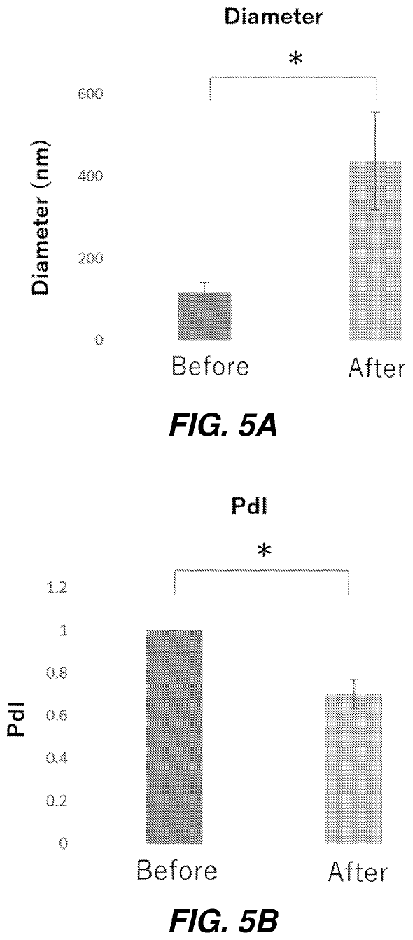

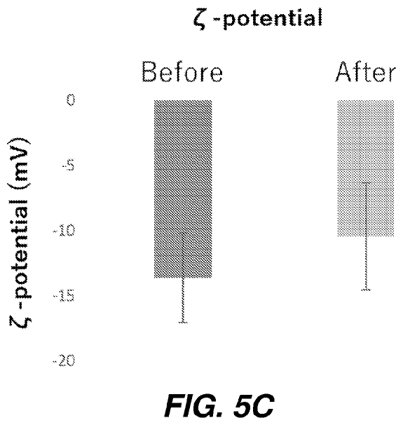

A comparison of physicochemical properties of PEG-coated second organelle complexes (2^{nd} OC; Q) before and after centrifugation was performed. Table 5 and FIGS. 5A-5C depict data related to diameter (FIG. 5A), PdI (FIG. 5B), and ζ-potential (FIG. 5C) before and after the centrifugation step.

TABLE 5
Comparison of Coated Second Organelle
Complexes Before & After Centrifugation

| | Diameter | | ζ-potential |
|---|---|---|---|
| 1 mM DMG-PEG | (nm) | PdI | (mV) |
| PEG-Q before CF | 118.1 ± 22.7 | 1.00 ± 0.00 | −13.6 ± 3.4 |
| step mt 100 μL:PEG | (peak 755.3 ± | | |
| 100 μL | 135.4) | | |
| PEG-Q after CF | 437.4 ± 119.5 | 0.70 ± 0.07 | −10.4 ± 4.1 |
| step mt 100 μL:PEG | (peak 794.3 ± | | |
| 100 μL | 120.2) | | |

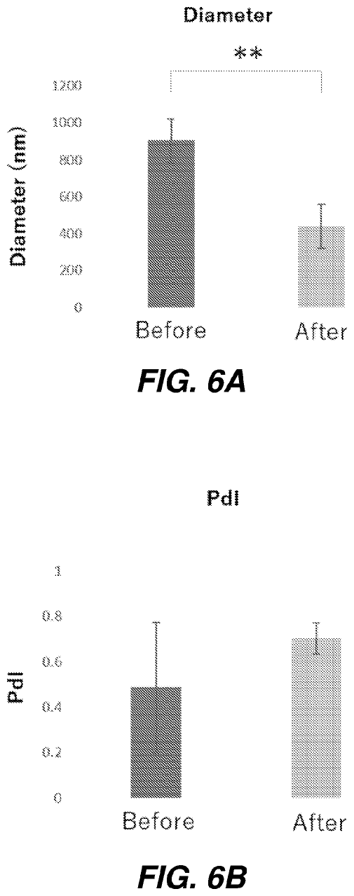

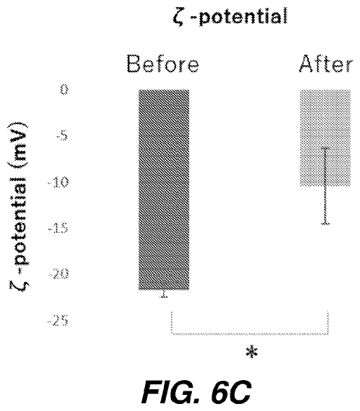

Next a comparison of physical properties between PEG-coated second organelle complexes (purified) and second organelle complexes (control) was performed. Table 6 and FIGS. 6A-6C depict data related to diameter (FIG. 6A), PdI (FIG. 6B), and ζ-potential (FIG. 6C) before and after PEG coating.

TABLE 6
Comparison of PEG-coated and Control
Second Organelle Complexes

| | Diameter | | ζ-potential |
|---|---|---|---|
| 1 mM DMG-PEG | (nm) | PdI | (mV) |
| Q | 901.8 ± 116.2 | 0.49 ± 0.29 | −21.7 ± 0.8 |
| mt 100 μL:tris | (peak 1368 ± 949.2) | | |
| 100 μL | | | |
| PEG-Q | 437.4 ± 119.5 | 0.70 ± 0.07 | −10.4 ± 4.1 |
| mt 100 μL:PEG | (peak 794.3 ± 120.2) | | |
| 100 μL | | | |

### Evaluation of Different PEG Coating Conditions

The coating of PEG was attempted with different temperatures and post-coating centrifugation conditions. The mitochondrial concentration was changed from 0.1 mg/mL to 1 mg/mL. Additionally, the PEG coating temperature was changed from room temperature to 4° C. Furthermore, the centrifugal speed was changed from 10,000 g to 3,000 g and the resuspension volume was changed from 100 μL to 1,000 μL. 1 mM DMG-PEG (100 μL) was added to a tube comprising 1 mg/mL Mitochondria (100 μL). After pipetting 3 times with a wide-mouth tip, an incubation step was performed at 4° C. for 15 min for PEG-Lipid anchoring. Following the incubation, centrifugation was performed at 4° C. for 10 min at 3,000 g for removing micelles. Following centrifugation, the pellet comprises PEG coated mitochondria and the supernatant comprises PEG-lipid micelles. The supernatant was removed and the pellet was resuspended with Tris buffer. The reproducibility of physical properties of PEG coated mitochondria was confirmed with the above protocol.

### Evaluation of PEG Amount

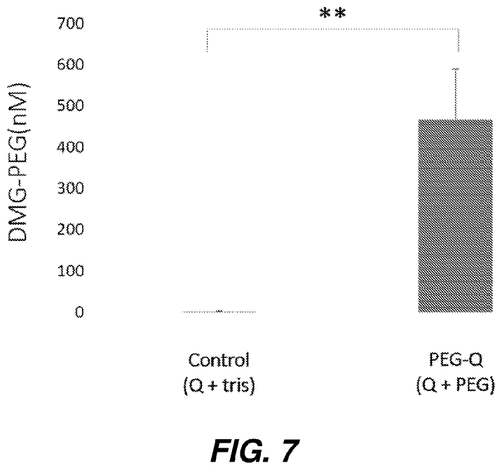

The determination of the amount of PEG incorporated was performed using ELISA. PEG content was measured using ELIZA Kit (ENZO PEGylated protein ELISA kit) (competitive assay). The amount of PEG coated in mitochondria was estimated. FIG. 7 depicts data related to PEG content of control second organelle complexes (Q+tris) and coated second organelle complexes (Q+PEG). The following set of calculations was performed: PEG wt/Q wt→466 nM (50 μL)/0.1 mg/1000 μL (50 μL)→466×10⁻⁶ nmol/μL (50 μL)/0.1 mg/20→50×466×10⁻⁶ nmol/5×10⁻³ mg→23.3×10⁻³ nmol/5×10⁻³ mg→4.66 nmol/mg→2489×4.66 ng/mg→11598.74 ng/mg→about 10 μg/mg. The amount of PEG anchoring into mitochondria was determined to be about 10 μg PEG/1 mg mitochondria, and therefore 1% of given PEG was anchored into mitochondria by mass.

### Evaluation of Mitochondrial Function

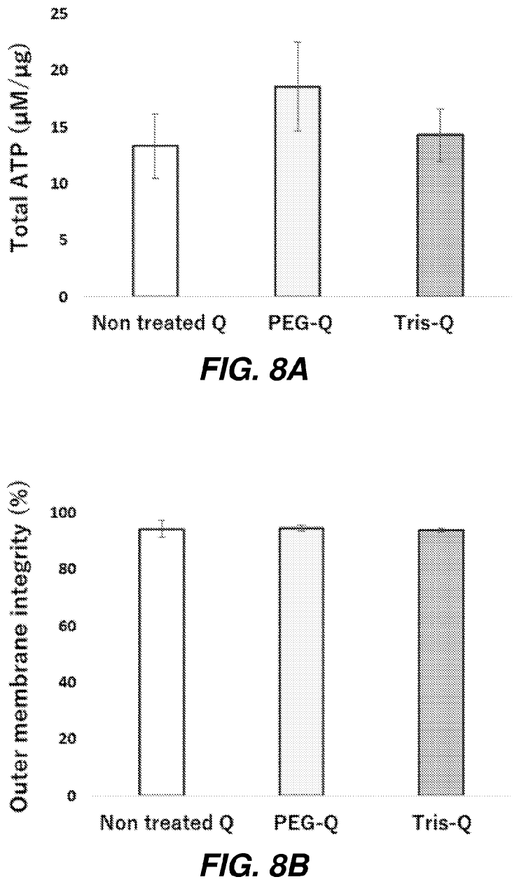

Mitochondrial function was evaluated in intact second organelle complexes (untreated Q), PEG coated second organelle complexes (PEG-Q), and control second organelle complexes (Tris-Q). Control second organelle complexes received tris buffer instead of PEG-lipid and underwent centrifugation, supernatant removal, and resuspension. FIGS. 8A-8B depict data related to ATP level (FIG. 8A) and outer membrane integrity (FIG. 8B) for intact second organelle complexes (untreated Q), PEG coated second organelle complexes (PEG-Q), and control second organelle complexes (Tris-Q). It was determined that PEG coating does not reduce mitochondrial function.

### Investigation of Peptide Modification of PEGylated Second Organelle Complexes

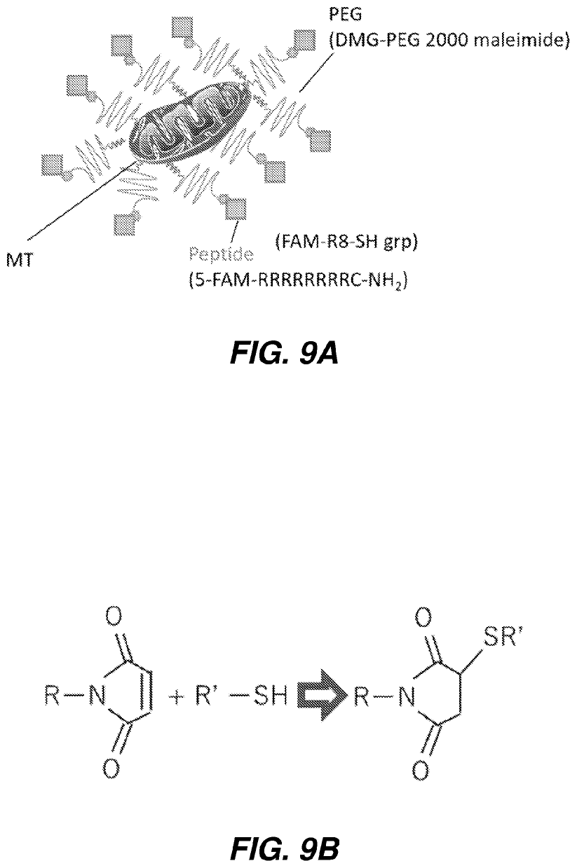

FIGS. 9A-9B depict non-limiting exemplary schematics showing a PEGylated mitochondrion wherein the PEG-lipid conjugate is peptide modified (FIG. 9A) and a maleimide (mal) group attached to PEG (FIG. 9B). A peptide (e.g., 5-FAM-RRRRRRRRC-NH₂) can be added to the lipid-polymer conjugate with the methods provided herein. A specific chemical reaction between the maleimide function group at the PEG and the SH group in the peptide can be employed to add a functional element (e.g., R8). Conditions including temperature, time, and amount of peptide modification during reaction were examined. The physical properties of peptide-treated second organelle complexes were evaluated, and second organelle complexes treated with peptide were evaluated by FACS.

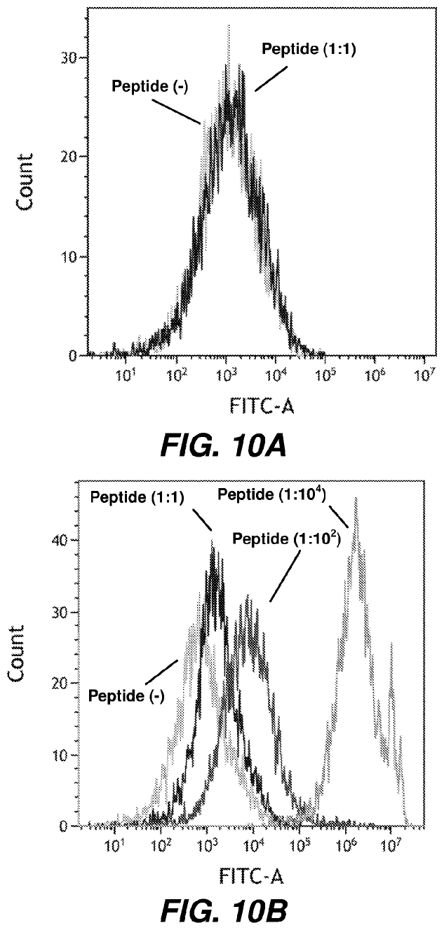

Peptide modification of second organelle complexes at 25° C. for 1 hr was first investigated. Tables 7-8 and FIGS. 10A-10B depict data related to uncoated second organelle complexes (Q) reacted in presence/absence of peptide (FIG. 10A) and coated second organelle complexes (PEG (mal)-Q) reacted at the indicated PEG: peptide molar ratios (FIG. 10B) at 25° C. for 1 hr. No peptide modification was observed without PEG (mal). However, peptide modification was observed in PEG (mal) Q (coated second organelle complexes). Additionally, the higher the dose (mol ratio) of peptide used, the more peptide modification was observed.

TABLE 7
Peptide Modification in Absence of PEG coating

| 25° C. 1 hr | | Diameter | | ζ-potential |
|---|---|---|---|---|
| Modification | n | (nm) | PdI | (mV) |
| Q | | | | |
| Peptide (−) | 1 | 641.2 | 0.63 | −15.6 |
| Peptide (+) | 1 | 718.7 | 0.69 | −15.5 |

TABLE 8
Peptide Modification in Presence of PEG coating at 25° C.

| 25° C. 1 hr | | Diameter | | ζ-potential |
|---|---|---|---|---|
| modification | n | (nm) | PdI | (mV) |
| PEG (mal)-Q | | | | |
| Peptide (−) | 1 | 640.6 | 0.69 | −10.8 |

| | |
|---|---|
| Peptide (+) | (PEG:Peptide(mol ratio)) |

| | | | | |
|---|---|---|---|---|
| 1:1 | 1 | 806.0 | 0.62 | −10.2 |
| 1:10² | 1 | 719.7 | 0.69 | −9.0 |
| 1:10⁴ | 1 | 1160 | 0.63 | −8.3 |

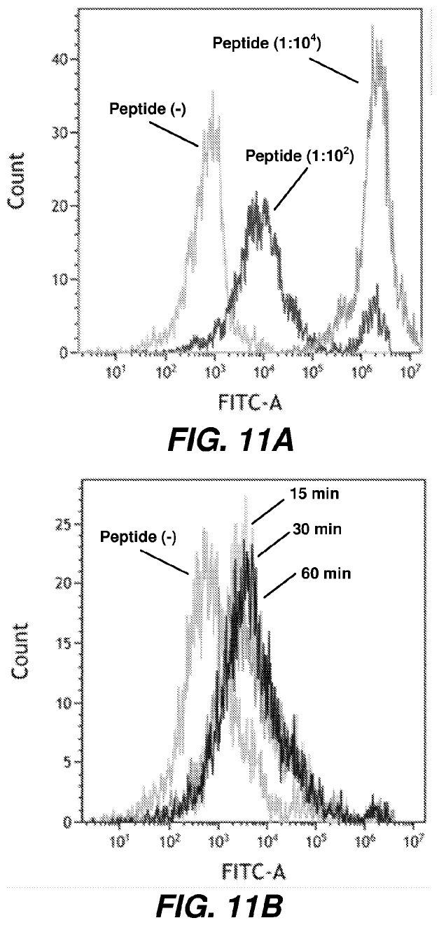

Peptide modification of PEGylated second organelle complexes at 4° C. for 15 min to 1 hr was next investigated. Tables 9-10 and FIGS. 11A-11B depict data related to peptide modification of second organelle complexes at 4° C. FIG. 11A depicts coated second organelle complexes (PEG (mal)-Q) reacted at indicated PEG: peptide molar ratios. FIG. 11B depicts coated second organelle complexes (PEG (mal)-Q) reacted with peptide for the indicated time period or without peptide. Peptide modification did occur when the reaction was performed at 4° C. Additionally, the higher the dose (mol ratio) of peptide used, the more peptide modification was observed at 4° C. However, it was found that adjusting the peptide modification time from 15 min to 1 hr did not impact modification amount at 4° C.

TABLE 9
Peptide Modification in Presence of PEG coating at 4° C.

| 4° C. 1 hr | | Diameter | | ζ-potential |
|---|---|---|---|---|
| modification | n | (nm) | PdI | (mV) |
| PEG(mal)-Q | | | | |
| Peptide (−) | 1 | 784 | 0.28 | −9.0 |

| | |
|---|---|
| Peptide (+) | (PEG:peptide(mol ratio)) |

| | | | | |
|---|---|---|---|---|
| 1:10² | 1 | 1053 | 0.06 | −10.2 |
| 1:10⁴ | 1 | 1247 | 0.39 | −6.4 |

TABLE 10
Peptide Modification in Presence of PEG coating at 4° C.

| 4° C. 15 min-1 hr | | Diameter | | ζ-potential |
|---|---|---|---|---|
| modification | n | (nm) | PdI | (mV) |
| PEG(mal)-Q | | | | |
| Peptide (−) | 1 | 1026 | 0.17 | −11.6 |
| Peptide (+) | 1 | 1081 | 0.37 | −9.6 |

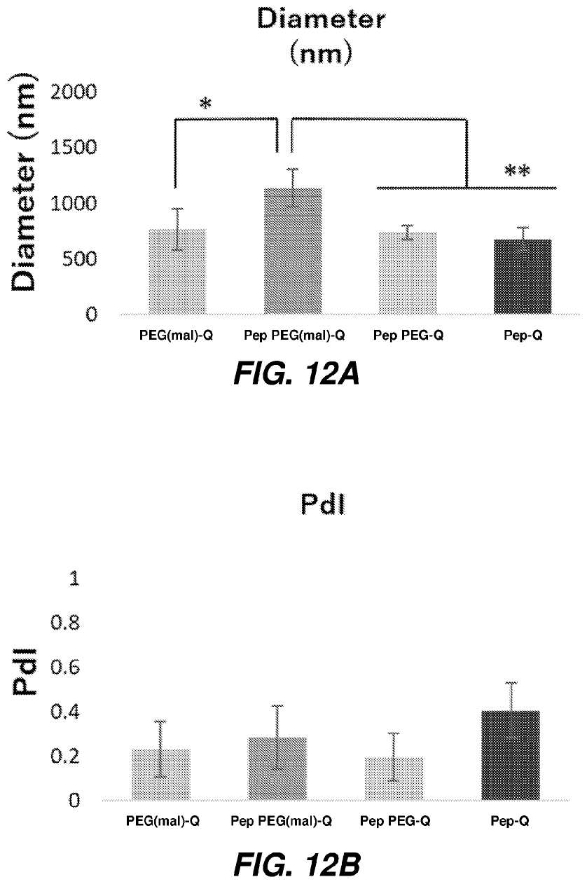

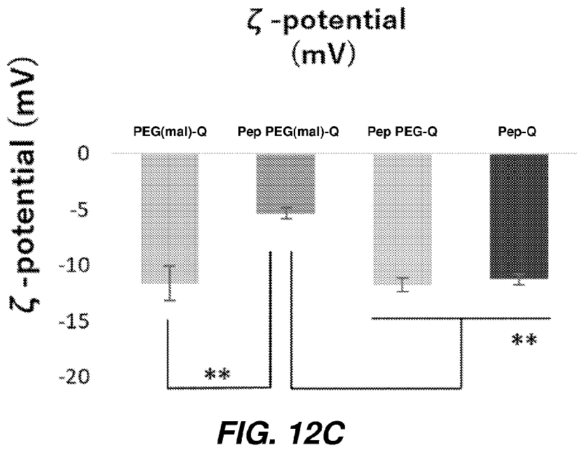

The physical properties of peptide-modified PEG-Q were investigated next. Peptide modification was performed at a PEG: Peptide mol ratio of 1:10⁴ at 4° C. for 15 min. Table 11 and FIGS. 12A-12C depict data related to depict data related to diameter (FIG. 12A), PdI (FIG. 12B), and ζ-potential (FIG. 12C) of PEG(mal)-Q (DMG-PEG 2000 maleimide+Q), Pep PEG(mal)-Q (Peptide+DMG-PEG 2000 maleimide+Q), Pep PEG-Q (Peptide+DMG-PEG 2000+Q), and Pep-Q (Peptide+Q). Zeta potential was found to shift to neutral after addition of peptide.

TABLE 11
Physical Properties of Peptide-modified PEG-Q

| | Diameter | | ζ-potential |
|---|---|---|---|
| | (nm) | PdI | (mV) |
| PEG(mal)-Q: DMG-PEG 2000 | 763.7 ± 183.5 | 0.23 ± 0.12 | −11.6 ± 1.5 |
| maleimide + Q | | | |
| Pep PEG(mal)-Q: Peptide + | 1140.7 ± 167.4 | 0.28 ± 0.14 | −5.3 ± 0.5 |
| DMG-PEG 2000 maleimide + Q | | | |
| Pep PEG-Q: Peptide + | 737.8 ± 58.8 | 0.20 ± 0.11 | −11.7 ± 0.6 |
| DMG-PEG 2000 + Q | | | |
| Pep-Q: Peptide + Q | 678.9 ± 100.1 | 0.41 ± 0.12 | −11.2 ± 0.5 |

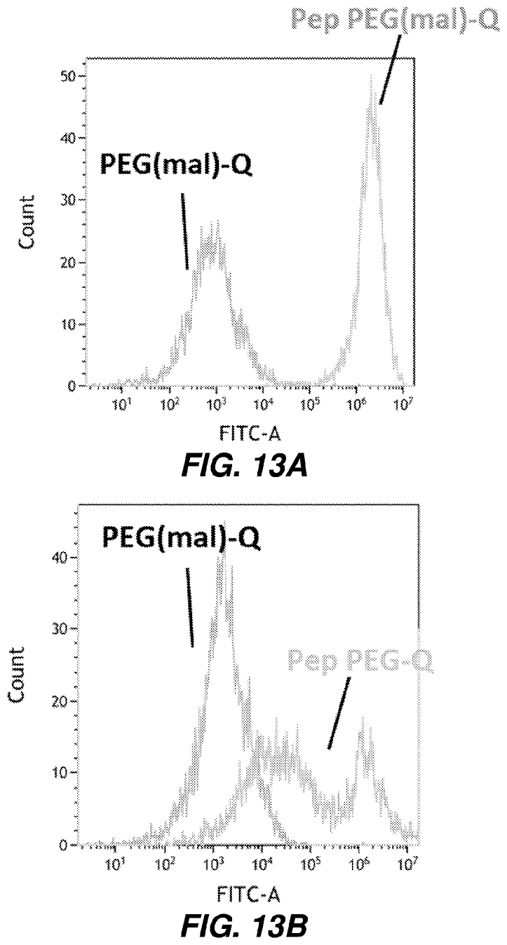

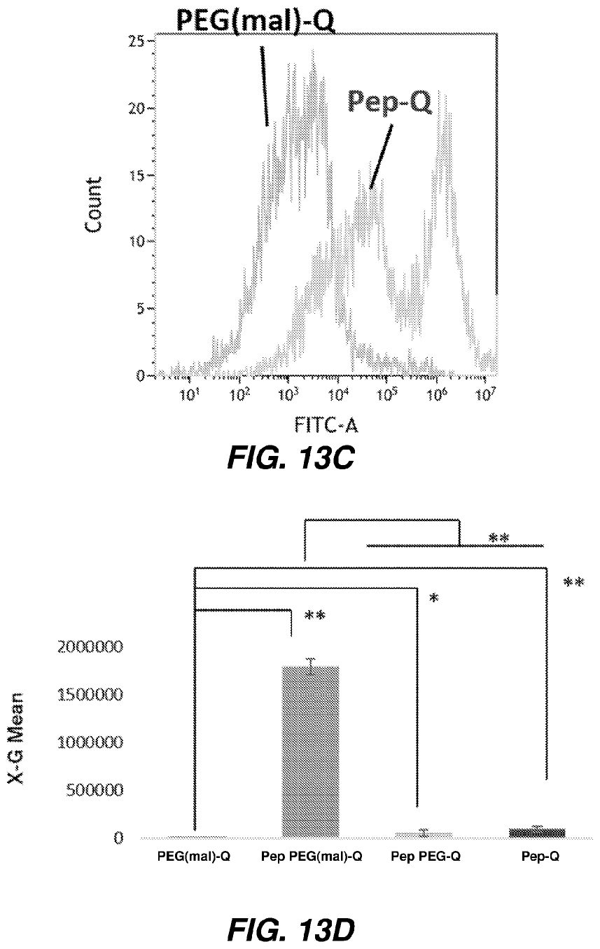

FAM-conjugated Peptide was employed and detection of FAM (fluorescence) in second organelle complexes treated with PEG (mal)+Peptide was performed. Table 12 and FIGS. 13A-13D depict FACS data related to PEG(mal)-Q (DMG-PEG 2000 maleimide+Q) versus Pep PEG(mal)-Q (Peptide+DMG-PEG 2000 maleimide+Q) (FIG. 13A), PEG(mal)-Q versus Pep PEG-Q (Peptide+DMG-PEG 2000+Q) (FIG. 13B), PEG(mal)-Q versus Pep-Q (Peptide+Q) (FIG. 13C), and all four modification conditions (FIG. 13D).

TABLE 12
Evaluation of Peptide-modified PEG-Q

| | X-G Mean |
|---|---|
| PEG(mal)-Q | 1397.2 ± 586.5 |
| Pep PEG(mal)-Q | 1796431.6 ± 82046.3 |
| Pep PEG-Q | 53331.5 ± 31462.3 |
| Pep Q | 94451.7 ± 28265.4 |

Evaluation of Uptake into HEK Cells

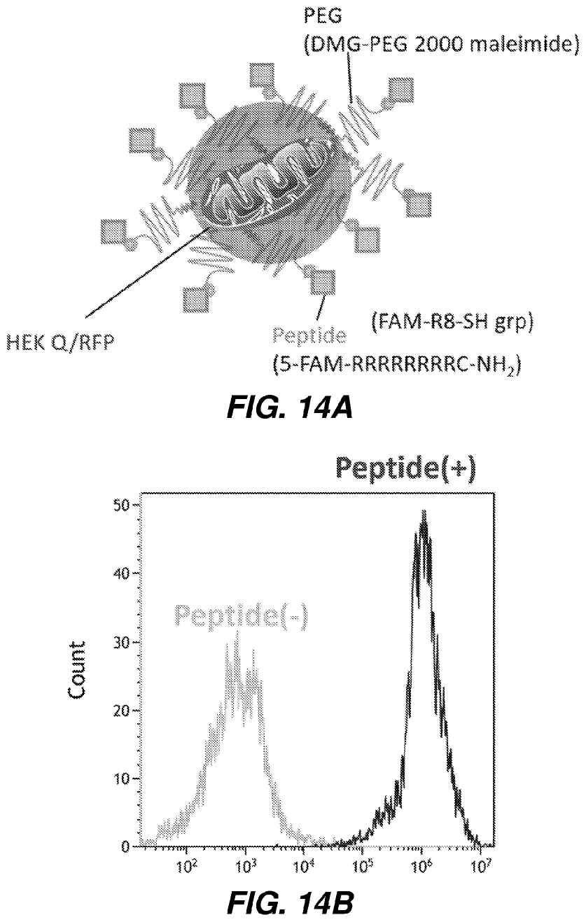

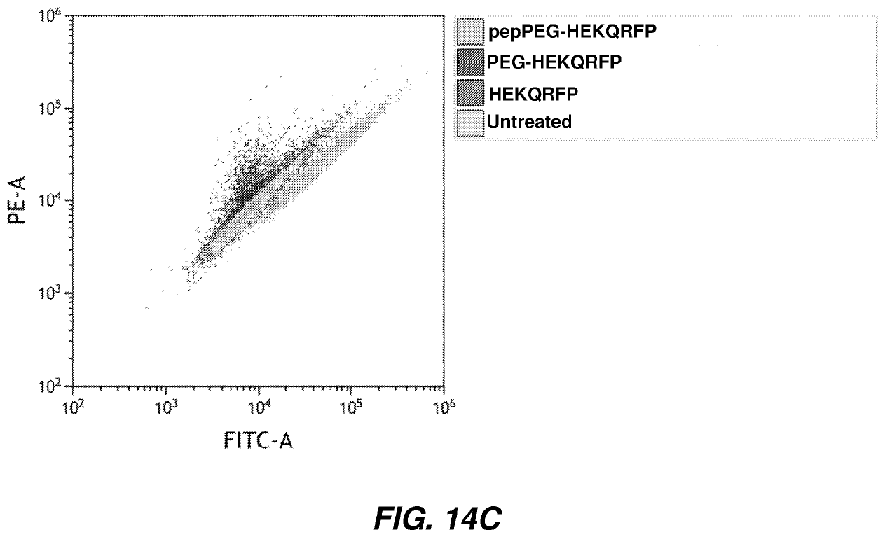

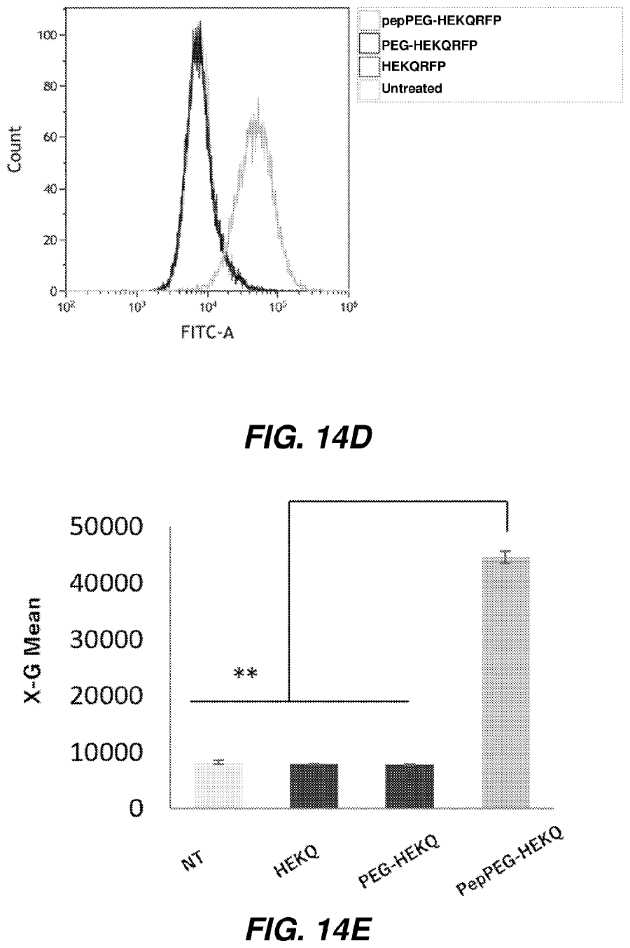

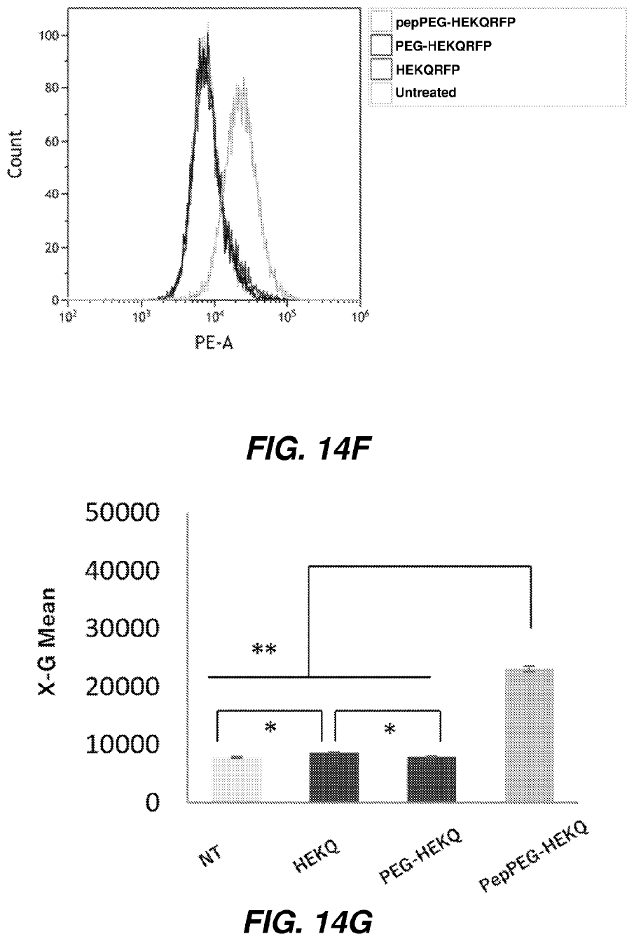

Tables 13-15 and FIGS. 14A-14G depict a schematic (FIG. 14A) and data (FIGS. 14B-14G) related to uptake of Peptide+PEG-coated HEKQ by HEK cells. nrANOVA followed by SNK test. It was first evaluated if Q (HEKQ/RFP) derived from RFP-labeled mitochondria in HEK cells can be coated with PEG and peptide. Peptide modification was performed at a PEG: Peptide mol ratio of 1:10⁴ at 4° C. for 15 min. FIG. 14B depicts FACS data related to Peptide(−) versus Peptide(+) modification conditions with HEKQ/RFP.

TABLE 13
PEG-coated HEKQ/RFP

| | | Diameter | | ζ-potential |
|---|---|---|---|---|
| | n | (nm) | PdI | (mV) |
| Peptide (−) | 1 | 547 | 0.58 | −8.3 |
| Peptide (+) | 1 | 772 | 0.44 | −2.3 |

Next HEKQ/RFP was coated with PEG and peptide and the uptake into HEK cells was evaluated (in FBS(+) media 6 hr after addition). FIG. 14C depicts FACS data related to pepPEG-HEKQRFP (PEG+peptide HEKQ/RFP), PEG-HEKQRFP (PEG HEKQ/RFP), HEKQRFP (HEKQ/RFP) and non-treated (NT).

TABLE 14
HEKQ/RFP

| | | Diameter | | ζ-Potential |
|---|---|---|---|---|
| | n | (nm) | PdI | (mV) |
| HEK Q/RFP | 1 | 404 | 0.11 | −8.2 |

TABLE 15
PEG coated HEKQ/RFP

| | | Diameter | | ζ-Potential |
|---|---|---|---|---|
| | n | (nm) | PdI | (mV) |
| Peptide (−) | 1 | 545 | 0.33 | −7.0 |
| Peptide (+) | 1 | 602 | 0.24 | −6.3 |

FIGS. 14D-14E depict FITC-A (FAM) data and FIGS. 14F-14G depict PE-A (RFP) FACS data. It was found that Peptide+PEG-coated HEKQ was significantly taken up by HEK cells. Additionally, it was observed that uncoated HEKQ is also taken up, but in smaller amounts. In contrast, PEG-coated HEKQ was not taken up.

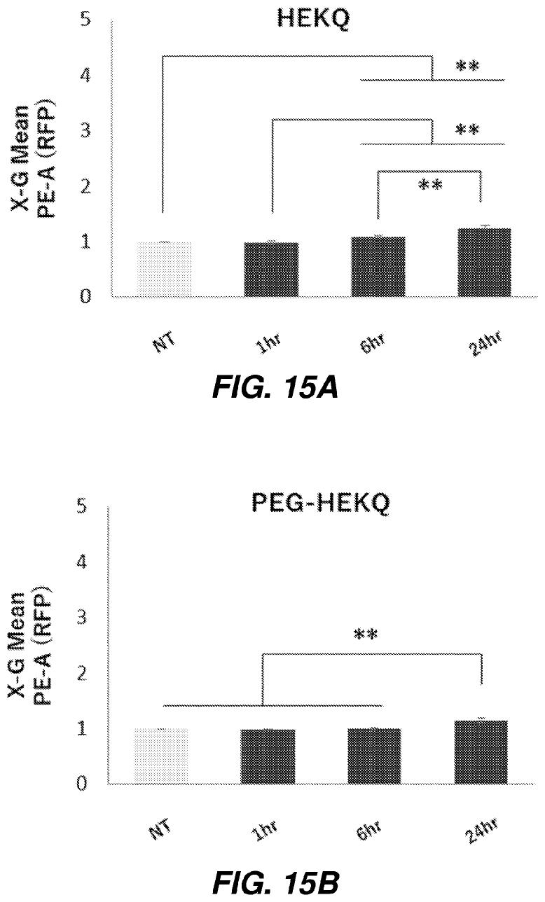

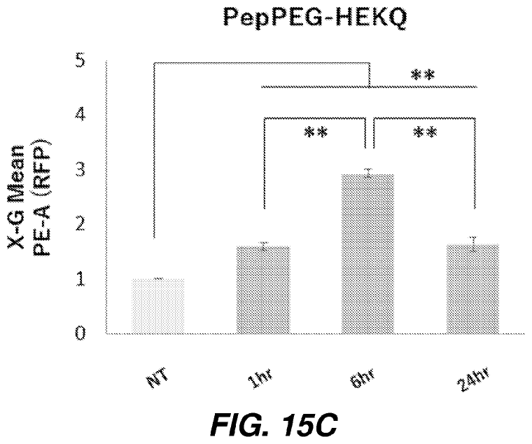

An investigation of the effect of incubation time on peptide-modified PEG-Q was next performed. FIGS. 15A-15C depict data related to the impact of incubation time on uptake of HEKQ (FIG. 15A), PEG-HEKQ (FIG. 15B), and PepPEG-HEKQ (FIG. 15C). The X-G Mean value of PE-A (RFP) was calculated by untreated (NT) as 1 relative to each other. Pep PEG-HEKQ uptake increased with longer dosing time at 6 hr, but the X-G Mean value decreased at 24 hr. HEKQ uptake increased with longer dosing time and PEG-HEKQ showed increased uptake after 24 hours of administration.

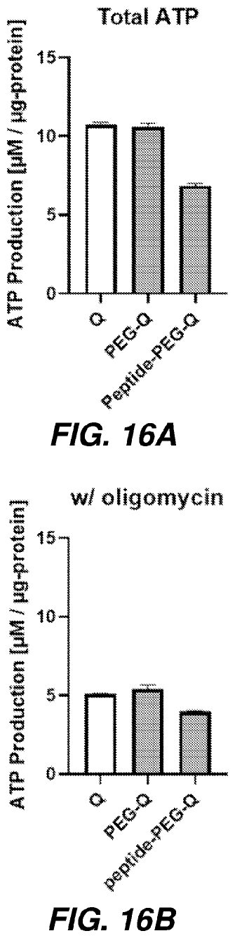

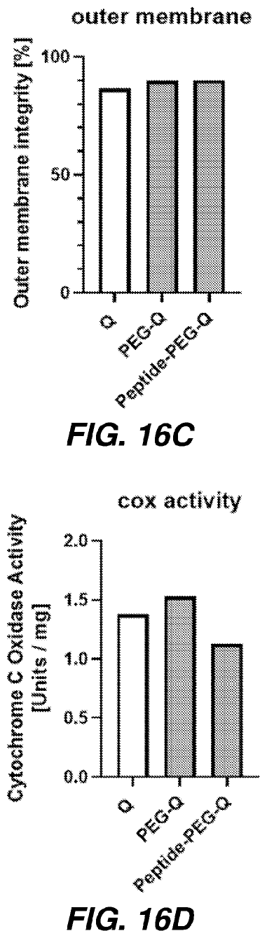

Finally an evaluation of the mitochondrial function of peptide-modified PEG-Q was performed. 0.5 mg protein was used to measure ATP and 1 mg protein were used to measure outer membrane. Protein levels were determined by the Pierce method and found to be as follows: Q, 77.6 μg/mL; PEG-Q, 77.6 μg/mL; Peptide-PEG-Q, 103.3 μg/mL. FIGS. 16A-16D depict data related to the mitochondrial function of peptide-modified PEG-Q with regards to ATP production (FIGS. 16A-16B), outer membrane integrity (FIG. 16C), and Cytochrome C Oxidase activity (FIG. 16D). It was found that peptide modification does not reduce mitochondrial function. ATP appears to be decreased with peptide-modified PEG-Q because the protein value used for protein correction is higher due to the effect of peptide.

### Evaluation of R8-PEG-HEKQ

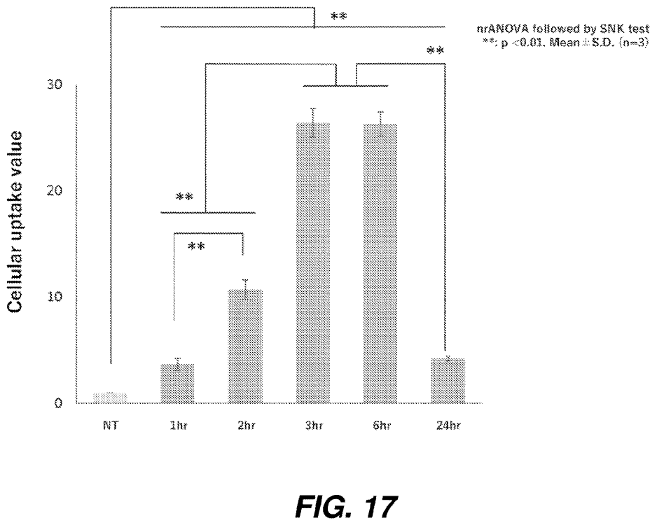

FIG. 17 depicts data related to uptake of Peptide (R8)-modified PEG-coated HEKQ by HEK cells (“R8-PEG-HEKQ”). The uptake of R8-PEG-HEKQ into HEK cells was examined at each dosing time using flow cytometer. The HEKQ contained red fluorescent protein (RFP), and geometric mean values of RFP (cellular uptake value) at each dosing time (1 hr, 2 hr, 3 hr, 6 hr, and 24 hr) relative to NT (Non treatment) as 1, was calculated. As shown in FIG. 17, R8-PEG-HEKQ uptake increased over time, reaching a maximum at 3 hr and 6 hr, but the geometric mean decreased at 24 hr.

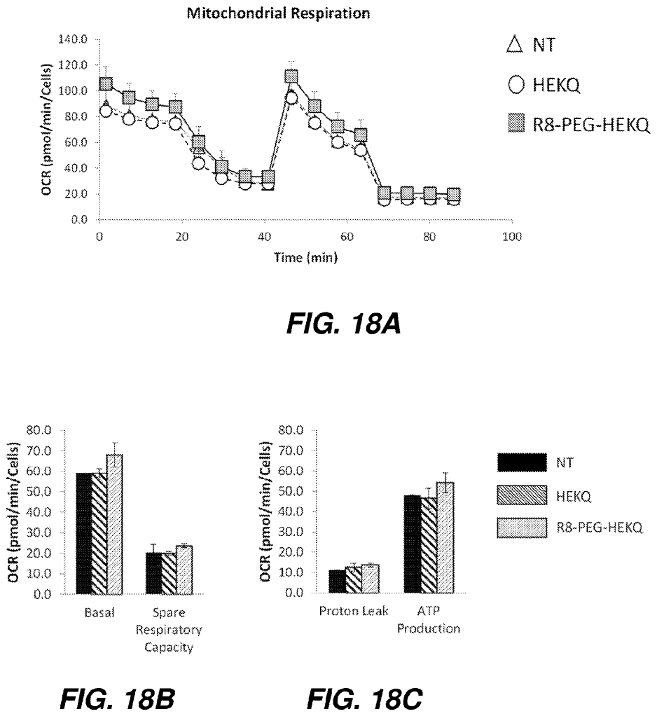

The effect of R8-PEG-HEKQ administration on Oxygen consumption rate (OCR) was tested in Flax analyzer (Seahorse assay). Cell seeding volume was 1.6×10⁴ cells/90 μL/well, and HEKQ (unmodified) or R8-PEG-HEKQ equivalent to 10% of the medium was administered for 24 hours (theoretical mt concentration after administration was 0.01 mg/ml). The overall results are shown in FIG. 18A, while each of basal respiration rate, spare respiratory capacity rate, proton leak rate, and ATP production rate are shown in FIGS. 18B-18C.

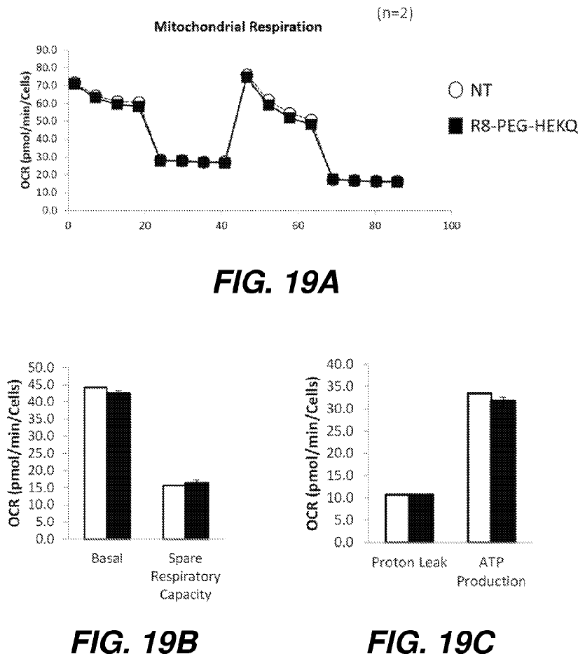

The effect of R8-PEG-HEKQ administration on Oxygen consumption rate (OCR) was tested in Flax analyzer (Seahorse assay). Cell seeding volume was 1.6×10⁴ cells/90 μL/well, and 0.1 mg/ml R8-PEG-HEKQ was administered for 24 hours. The overall results are shown in FIG. 19A-C, and R8-PEG-HEKQ does not increase OCR after 24 hours of treatment compared to no treatment (NT).

### Evaluation of R8-PEG-HEKQ

Using a method described herein, peptide (R8)-modified PEG-coated HeLaQ was prepared. By ultrafiltration, R8-PEG-HeLaQ (mt conc 1 mg/ml) and unmodified HeLaQ (mt conc 1 mg/ml) were prepared.

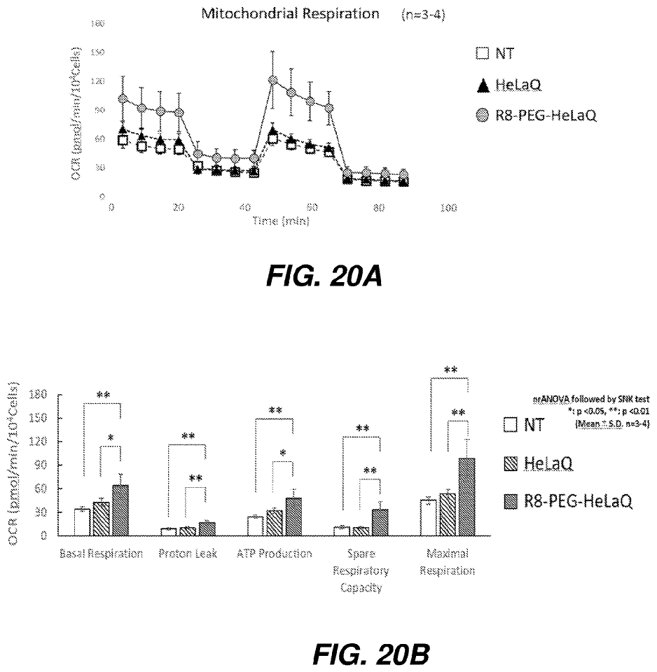

The effect of R8-PEG-HeLaQ administration on Oxygen consumption rate (OCR) was tested in Seahorse XF HS Mini Analyzer. HEK cell seeding volume was 0.9×10⁴ cells/90 μL/well. R8-PEG-HeLaQ and unmodified HeLaQ was administered, at mt concentration of 0.1 mg/ml, for 24 hours. The overall results are shown in FIG. 20A, while each of basal respiration rate, spare respiratory capacity rate, proton leak rate, and ATP production rate are shown in FIG. 20B. As shown in FIGS. 20A-20B, administration of R8-PEG-HeLaQ significantly increased OCR of HEK cells, compared to both unmodified HeLaQ and blank (NT).

### Supplementary Materials and Methods

### Mitochondrial Particle Size Measurements and Charge Measurements

Particle sizes were measured in 50 μL of mitochondrial (PEG-coated and peptide-modified mitochondria) suspensions. The particle size measuring conditions were as follows: RI, 1.590; absorbance, 0.010; media, 250 mM Tris-sucrose buffer; temperature 25.0° C.; and visc. 1.1459 cP, RI 1.342. Charge measurements were conducted by resuspend 50 μL of the above suspension in 500 μL of buffer. The charge measuring conditions were as follows: RI, 1.590; absorbance, 0.010; buffer, 250 mM Tris-sucrose; temperature 25.0° C.; and visc. 1.1459 cP, RI 1.342, dielectric const. 78.5. A Zetasizer Nano ZS (Malvern) was employed in the measurements.

### Determination of the Amount of PEG Coating Using ELISA

An ENZO PEGylated protein ELISA kit (competition method) was employed in the analysis. A 96-well plate included in the kit was employed. The following steps were performed: (1) 50 μL/well of sample followed by 50 μL/well of Biotinylated PEG; (2) after gentle tapping the plate, shaking for 1 hour at room temperature; (3) after aspiration, wash 4 times with 300 μL/well of wash buffer; (4) PEG conjugate 100 μL/well followed by shaking for 30 minutes at RT; (5) after aspiration, wash 3 times with 300 μL/well of wash buffer; (6) TMB Substrate 100 L/well administration followed by shaking for 30 min at RT; (7) after administration of Stop Solution 2 (IN HCl) 100 μL/well; and (8) measure absorbance at 450 nm with a plate reader. PEG serial dilutions were made from 1 mM to 1 nM, and the same procedures as above were performed to obtain a calibration curve.

### ATP Production and Outer Membrane Integrity Assays

The ATP Assay was conducted under the following conditions: amount of mitochondrial solution added, 0.5 μg/well (protein quantification by Pierce method); reaction substrate, 10 mM Malate/5 mM Glutamate/0.1 mM ADP/10 mM Pi/70 mM KCl; and reaction conditions, room temperature for 10 min. For outer membrane integrity measurement, a Cytochrome C Oxidase assay was performed under the following conditions: amount of mitochondrial solution added, 3 μg/well (protein quantification by Pierce method); and reaction (measurement) temperature, 30° C. Outer membrane integrity was calculated employing the following equations (where ε=21.84, L=0.625):

$$Units / mg = ( Δ A ( w / Detergent ) ) / ( ε \times L \times applied protein [ mg ] ) ; and$$

$$Outer membrane integrity ( \% ) = ( Δ A ( w / Detergent ) - Δ A ( w / o Detergent ) ) / ( Δ A ( w / Detergent ) ) .$$

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A coated organelle complexes population,
wherein the coated organelle complexes comprise mitochondria and one or more of endoplasmic reticulum, peroxisomes, lysosomes, and Golgi apparatus,
wherein the surface of the coated organelle complexes comprises one or more lipid-polymer conjugates, and
wherein the one or more lipid-polymer conjugates comprise a polymer conjugated to a lipid.

2. The coated organelle complexes population of claim 1, wherein the polymer is a bio-soluble polymer and/or a biodegradable polymer.

3. The coated organelle complexes population of any one of claims 1-2, wherein the one or more lipid-polymer conjugates are 1,2-dimyristoyl-sn-glycerol, methoxy polyethylene glycol 2000 (DMG-PEG 2000) and/or distearoyl-sn-glycero-3-phosphoethanolamine-N—, methoxy polyethylene glycol 2000 (DSPE-PEG 2000).

4. The coated organelle complexes population of any one of claims 1-3, wherein the one or more lipid-polymer conjugates does not comprise triphenylphosphonium (TPP), cholesterol, oleic acid, or any combination thereof.

5. The coated organelle complexes population of any one of claims 1-4, wherein the coated organelle complexes comprise coated first organelle complexes, coated second organelle complexes, or a combination of coated first organelle complexes and coated second organelle complexes,
wherein the coated first organelle complexes and coated second organelle complexes are depleted of cytosolic macromolecules,
wherein coated first organelle complexes are derived from (i) frozen cells; (ii) floating cells; and/or (iii) cells contacted with a surfactant at a concentration at or above the critical micellar concentration (CMC) for the surfactant, and
wherein coated second organelle complexes are derived from (i) adherent cells; and/or (ii) cells contacted with a surfactant at a concentration below the critical micellar concentration (CMC) for the surfactant.

6. The coated organelle complexes population of any claim 5, wherein the cytosolic macromolecules comprise cytosolic proteins, wherein the abundance of one or more cytosolic proteins is depleted by at least about 90% as compared to the cells from which the organelle complexes population are derived, optionally the cytosolic proteins are p70S6K and/or glyceraldehyde 3-phosphate dehydrogenase (GAPDH).

7. The coated organelle complexes population of any one of claims 1-6, wherein the coated organelle complexes comprise:
one or more mitochondrial matrix proteins, optionally mitochondrial transcription factor A (TFAM) and/or citrate synthase (CS);
one or more outer mitochondrial membrane proteins, optionally outer mitochondrial membrane complex subunit 20 (TOMM20);
one or more lysosome proteins, optionally lysosomal-associated membrane protein 2 (LAMP2), mannose-6-phosphate receptor (M6PR), and/or lysosomal-associated membrane protein 1 (LAMP1);
one or more peroxisome proteins, optionally catalase and/or ATP-binding cassette transporter 1, subfamily D, type 3 (ABCD3);
one or more inner mitochondrial membrane proteins, optionally respiratory chain proteins;
mitochondrial DNA, mitochondrial RNA, or both;
one or more Golgi apparatus proteins, optionally Golgin-97, Sintaxin-6, TGOLN2/trans-Golgi network protein 2 (TGN46), Golgi matrix protein 130 (GM130), and/or Mannosidase Alpha Class 2A Member 1 (MAN2A1); and/or
one or more endoplasmic reticulum proteins, optionally Calreticulin and/or Calnexin.

8. The coated organelle complexes population of any one of claims 1-7, wherein the organelle complexes are derived from cells treated with a mitochondria-activating agent, optionally resveratrol.

9. The coated organelle complexes population of any one of claims 1-8, wherein the lipid comprises an amphipathic lipid having a hydrophobic moiety and a hydrophilic portion.

10. The coated organelle complexes population of claim 9, wherein the amphipathic lipid is selected from the group comprising phospholipids, aminolipids and sphingolipids.

11. The coated organelle complexes population of claim 10, wherein the phospholipid is selected from the group comprising dimyristoylphosphatidylglycerol (DMG), distearoyl phosphatidyl-ethanolamine (DSPE), dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2 Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroylphosphatidylglycerol (DLPG), diarachidoylphosphatidylglycerol (DAPG), dipalmitoylphosphatidylglycerol (DPPG), distearoylphosphatidylglycerol (DSPG), dioleoyl-phosphatidylglycerol (DOPG), dimyristoyl phosphatidic acid (DMPA), dipalmitoyl phosphatidic acid (DPPA), distearoyl phosphatidic acid (DSPA), diarachidoylphosphatidic acid (DAPA), dimyristoylphosphatidylethanolamine (DMPE), dipalmitoylphosphatidylethanolamine (DPPE), dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), and dioleoyl-phosphatidylinositol (DOPI).

12. The coated organelle complexes population of any one of claims 10-11, wherein the phospholipid comprises a saturated fatty acid with a C14-C20 carbon chain and/or an unsaturated fatty acid with a C14-C20 carbon chain.

13. The coated organelle complexes population of any one of claims 1-12, wherein the lipid comprises a phosphatidylethanolamine, and wherein the phosphatidylethanolamine:
has a carbon chain length of 10 to 20;
comprises saturated fatty acids;
comprises unsaturated fatty acids;
comprises saturated fatty acids and unsaturated fatty acids; and/or
is selected from the group comprising distearoylphosphatidyl-ethanolamine (DSPE), dimyristoylphosphatidylethanolamine (DMPE), dipalmitoylphosphatidylethanolamine (DPPE), and dioleoylphosphatidylethanolamine (DOPE).

14. The coated organelle complexes population of any one of claims 1-13, wherein the polymer has a molecular weight:
between about 100 and about 20000 daltons (Da);
between about 100 and about 1000 Da;
between about 1000 and about 3500 Da;
between about 3500 and 7000 Da; and/or
of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 Da.

15. The coated organelle complexes population of any one of claims 1-14, wherein the polymer is or comprises poly(acrylate), poly(methacrylate), poly(acrylic acid), poly(acrylamide), poly(vinylpyridine), poly(vinylpyrrolidone), poly(vinyl alcohol), a naturally-derived polymer, poly(ether), poly(maleic anhydride), poly(styrene sulfonate), poly(allylamine hydrochloride), poly(sulfone), poly(ethersulfone), poly(ethylene glycol), copolymers thereof, or any combination thereof.

16. The coated organelle complexes population of any one of claims 1-15, wherein the surface of the coated organelle complexes comprises the one or more lipid-polymer conjugates at a molar ratio of greater than at least about 0.1%, about 0.5%, about 1%, or about 5%, of the mass of the organelle complexes.

17. The coated organelle complexes population of any one of claims 1-16,
wherein the presence of the one or more lipid-polymer conjugate in the surface of the coated organelle complexes does not reduce mitochondrial function; or
wherein the presence of the one or more lipid-polymer conjugate in the surface of the coated organelle complexes reduces mitochondrial function less than about 10 percent, about 5 percent, or about 1 percent, as compared to organelle complexes not comprising the one or more lipid-polymer conjugates,
optionally mitochondrial function comprises one or more of ATP production, outer membrane structural integrity of mitochondria, and cytochrome c oxidase (COX) activity.

18. The coated organelle complexes population of any one of claims 1-17,
wherein the polydispersity index (PDI) of the coated organelle complexes population is within about 5 percent, about 10 percent, about 15 percent, or about 20 percent, of the PDI of a population of organelle complexes not comprising the one or more lipid-polymer conjugates;
wherein the ζ-potential of the coated organelle complexes population is at least about 5 percent, about 10 percent, about 25 percent, about 50 percent, about 75 percent, or about 100 percent, more positive as compared to the ζ-potential of a population of organelle complexes not comprising the one or more lipid-polymer conjugates; and/or
wherein the average diameter of the coated organelle complexes population is at least about 5 percent, about 10 percent, about 25 percent, about 50 percent, about 75 percent, or about 100 percent, smaller as compared to the average diameter of a population of organelle complexes not comprising the one or more lipid-polymer conjugates.

19. The coated organelle complexes population of any one of claims 1-18,
wherein the stability of the coated organelle complexes population in solution is at least about 5 percent, about 10 percent, about 25 percent, about 50 percent, about 75 percent, or about 100 percent, greater than a population of organelle complexes not comprising the one or more lipid-polymer conjugates;
wherein the physical stability of the coated organelle complexes population against internal stimuli and/or external stimuli is at least about 5 percent, about 10 percent, about 25 percent, about 50 percent, about 75 percent, or about 100 percent, greater as compared to a population of organelle complexes not comprising the one or more lipid-polymer conjugates; and/or
wherein at least about 70 percent, about 80 percent, about 90 percent, or about 100 percent, of the coated organelle complexes population is functional after the population undergoes one or more freeze-thaw cycles.

20. The coated organelle complexes population of any one of claims 1-19, wherein one or both ends of the polymer is functionalized with a functional group selected from the group comprising vinyl, carboxylate, hydroxyl, epoxide, sulfhydryl, amide, acrylate, thiol, azide, maleimide, isocyanate, aziridine, carbonate, N-hydroxysuccinimide ester, imidoester, carbodiimide, anhydride, succinimidyl carbonate, and amine, or any combination thereof.

21. The coated organelle complexes population of any one of claims 1-20, wherein the one or more lipid-polymer conjugates further comprise a targeting agent, optionally polyarginine.

22. The coated organelle complexes population of claim 21, wherein upon contact of the coated organelle complexes population with a population of cells, the coated organelle complexes have at least about 1.1-fold superior incorporation capability into said cells as compared to organelle complexes not comprising the one or more lipid-polymer conjugates comprising the targeting agent.

23. The coated organelle complexes population of claim 22, wherein the targeting agent is configured to bind a ligand on the surface of a target cell, optionally a target cell of a subject in need.

24. The coated organelle complexes population of claim 23, wherein the binding of the targeting agent and ligand causes the coated organelle complexes to be incorporated by the target cell.

25. The coated organelle complexes population of any one of claims 23-24, wherein the ligand is:
differentially expressed between target cells and non-target cells;
absent on non-target cells; and/or
over expressed on target cells.

26. The coated organelle complexes population of any one of claims 23-25, wherein the target cells are residents of a target tissue, wherein said target tissue is cancerous, inflamed, damaged, dysfunctional, infected, the site of disease or disorder, and/or proximate to a site of a disease or disorder, optionally the tissue comprises adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue.

27. The coated organelle complexes population of any one of claims 21-26, wherein the targeting agent is configured to bind axons.

28. The coated organelle complexes population of any one of claims 21-27, wherein the targeting agent is or comprises a peptide, an antigen binding domain, a cytokine, a chemokine, an aptamer, a growth factor, a hormone, a cytokine, an interleukin, a receptor, or any combination thereof.

29. The coated organelle complexes population of claim 28, wherein the antigen binding domain comprises an antibody, an antibody fragment, an scFv, a Fv, a Fab, a (Fab′)2, a single domain antibody (SDAB), a VH or VL domain, a camelid VHH domain, a Fab′, a F(ab′)2, a Fv, a scFv, a dsFv, a diabody, a triabody, a tetrabody, a multispecific antibody formed from antibody fragments, a single-domain antibody (sdAb), a single chain comprising anticomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

30. The coated organelle complexes population of any one of claims 1-29, wherein the one or more lipid-polymer conjugates further comprise a detectable moiety configured for detection of the coated organelle complexes in vivo and/or in vitro.

31. The coated organelle complexes population of claim 30, wherein the detectable moiety comprises a fluorescent molecule, optionally selected from the group comprising fluorescein amidite (FAM), fluorescein dyes, carbocyanine, merocyanine, styryl dyes, oxonol dyes, phycoerythrin, erythrosin, eosin, rhodamine dyes, coumarin, coumarin dyes, Oregon Green Dyes, Texas Red, Texas Red-X, Spectrum Red™, Spectrum Green™, cyanine dyes, Fluor dyes, BODIPY dyes, derivatives thereof, or any combination thereof.

32. The coated organelle complexes population of any one of claims 30-31, wherein the detectable moiety comprises a fluorescent protein, optionally selected from the group comprising green fluorescent protein (GFP), enhanced GFP (EGFP), blue fluorescent proteins (BFP), cyan fluorescent protein (CFP), yellow fluorescent protein (YFP), red fluorescent protein (RFP), derivatives thereof, or any combination thereof.

33. The coated organelle complexes population of any one of claims 30-32, wherein the detectable moiety comprises a radioisotope that is detectable by Single Photon Emission Computed Tomography (SPECT) and/or Position Emission Tomography (PET), optionally selected from the group comprising iodine-131 (¹³¹I), iodine-125 (¹²⁵I), bismuth-212 (²¹²Bi), bismuth-213 (²¹³Bi), astatine-221 (²¹¹At), copper-67 (⁶⁷Cu), copper-64 (⁶⁴Cu), rhenium-186 (¹⁸⁶Re), rhenium-188 (¹⁸⁸Re), phosphorus-32 (³²P), samarium-153 (¹⁵³Sm), lutetium-177 (¹⁷⁷Lu), technetium-99m (^{99m}Tc), gallium-67 (⁶⁷Ga), indium-111 (¹¹¹In), and thallium-201 (²⁰¹Tl).

34. The coated organelle complexes population of any one of claims 30-33, wherein the detectable moiety comprises a quantum dot (Qdot) fluorescent particle, optionally selected from the group comprising Qdot525, Qdot565, Qdot585, Qdot605, Qdot625, Qdot655, Qdot705, Qdot800, derivatives thereof, or any combination thereof.

35. The coated organelle complexes population of any one of claims 1-34, wherein the one or more lipid-polymer conjugates further comprise one or more secondary agents, optionally a therapeutic agent, further optionally a small molecule drug.

36. The coated organelle complexes population of claim 35, wherein one or more secondary agents is an anti-cancer agent, an anti-inflammatory agent, an anti-infective agent, a regenerative agent, a relaxing agent, an apoptosis-inhibiting agent, an apoptosis-inducing agent, an anti-coagulatory agent, an antioxidant molecule, an autophagy-inducing agent, a dermatological agent, a growth-stimulating agent, a vasodilating agent, a vasoconstricting agent, an analgesic agent, and an anti-allergic agent, condensate modifying drugs (c-MODS) or a combination thereof.

37. The coated organelle complexes population of any one of claims 35-36, wherein one or more secondary agents is a chemotherapeutic, a nucleic acid, a polysaccharide, a peptide, a polypeptide, or any combination thereof.

38. The coated organelle complexes population of any one of claims 35-37, wherein one or more secondary agents is a protein phosphatase inhibitor, a kinase inhibitor, a cytokine, an inhibitor of an immune inhibitory molecule, an immune modulator, an anti-metastatic, a chemotherapeutic, a hormone or a growth factor antagonist, an alkylating agent, a TLR agonist, a cytokine antagonist, a cytokine antagonist, or any combination thereof.

39. The coated organelle complexes population of any one of claims 35-38, wherein one or more secondary agents is an agonistic or antagonistic antibody specific to a checkpoint inhibitor or checkpoint stimulator molecule such as PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3.

40. A method for generating a coated organelle complexes population, the method comprising:
contacting organelle complexes in a first solution with one or more lipid-polymer conjugates to generate coated organelle complexes; and
recovering coated organelle complexes from the first solution to generate the coated organelle complexes population.

41. The method of claim 40, wherein the method further comprises:
incubating the first solution after the contacting step,
optionally for about 1 minute to about 120 minutes, further optionally for about 15 minutes.

42. The method of any one of claims 40-41, wherein the contacting step comprises applying a physical stimulus to the first solution, optionally shaking, mixing and/or stirring, further optionally pipetting.

43. The method of any one of claims 40-42, wherein the incubating step is performed at a first temperature.

44. The method of any one of claims 40-43, wherein the organelle complexes are present in the first solution at concentration of about 0.01 mg/mL to about 10 mg/mL further optionally about 0.1 mg/mL to about 1 mg/mL, optionally about 1 mg/mL.

45. The method of any one of claims 40-44, wherein the contacting step comprises contacting the first solution with about 1 μL to about 1000 μL of a solution comprising the one or more lipid-polymer conjugates, and wherein the one or more lipid-polymer conjugates are present at a concentration of about 0.1 mM to about 10 mM, optionally a 1 mM 100 μL solution.

46. The method of any one of claims 40-45, wherein recovering the coated organelle complexes from the first solution comprises one or more centrifugation steps.

47. The method of any one of claims 40-46, wherein recovering the coated organelle complexes from the first solution comprises:
centrifuging the first solution at a first centrifugal force;
collecting the pellet to recover the coated organelle complexes.

48. The method of any one of claims 40-47, wherein the first centrifugal force is about 100 g to about 10000 g, optionally the first centrifugal force is about 3000 g.

49. The method of any one of claims 40-48, wherein collecting the pellet comprises resuspending the coated organelle complexes population in a second solution, optionally the second solution has a volume of about 50 μL to about 50 mL, further optionally about 100 μL to about 1000 μL, optionally about 1000 μL.

50. The method of any one of claims 40-49, wherein centrifuging step is performed for at least about 1 min, about 2 min, about 3 min, about 4 min, about 5 min, about 6 min, about 7 min, about 8 min, about 9 min, about 10 min, about 15 min, or about 20 min.

51. The method of any one of claims 40-50, wherein the centrifugation step is performed at a second temperature.

52. The method of any one of claims 40-51, wherein the first temperature and/or the second temperature is about 0° C. to about 50° C., optionally the first temperature is about 20° C. to about 25° C. and the second temperature is about 0° C. to about 4° C.

53. The method of any one of claims 40-52, wherein the recovering step depletes the one or more lipid-polymer conjugates that are not incorporated into the surface of the organelle complexes, optionally the one or more lipid-polymer conjugates that are not incorporated into the surface of the organelle complexes are present as micelles.

54. A coated organelle complexes population obtained by the method according to any one of claims 40-53.

55. A method for introducing coated organelle complexes into host cells, comprising:
contacting the coated organelle complexes population of any one of claims of 1-39 or 54 with a population of host cells,
wherein upon contact of the coated organelle complexes with the host cells, the coated organelle complexes are capable of incorporating into the host cells.

56. The method of claim 55, wherein the host cells comprise one or more mammalian cells selected from the group comprising an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof, optionally the stem cell comprises an embryonic stem cell, an induced pluripotent stem cell (iPSC), a hematopoietic stem/progenitor cell (HSPC), or any combination thereof.

57. The method of any one of claims 55-56, wherein the host cells are the cell of a subject, optionally a subject suffering from a disease or disorder, further optionally the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof.

58. The method of any one of claims 55-57, wherein the contacting is performed for a period time of at least about 1 hr, about 2 hr, about 3 hr, about 4 hr, about 5 hr, about 6 hr, about 7 hr, about 8 hr, about 9 hr, about 10 hr, about 12 hr, about 16 hr, about 20 hr, or about 24 hr.

59. The method of any one of claims 55-58,
wherein the method comprises attaching a targeting agent, a detectable moiety, and/or one or more secondary agents to the one or more lipid-polymer conjugates, and
wherein the polymer, the targeting agent, the detectable moiety, and/or the one or more secondary agents is functionalized with a functional group selected from the group comprising vinyl, carboxylate, hydroxyl, epoxide, sulfhydryl, amide, acrylate, thiol, azide, maleimide, isocyanate, aziridine, carbonate, N-hydroxysuccinimide, ester, imidoester, carbodiimide, anhydride, succinimidyl carbonate, and amine, and combinations thereof,
optionally the attaching comprise contacting the targeting agent, the detectable moiety, and/or the one or more secondary agents with the one or more lipid-polymer conjugates for at least about 1 min, about 2 min, about 3 min, about 4 min, about 5 min, about 6 min, about 7 min, about 8 min, about 9 min, about 10 min, about 15 min, about 20 min, about 30 min, about 40 min, about 50 min, or about 60 min, optionally at a temperature of about 0° C. to about 50° C., optionally about 20° C. to about 25° C. or about 0° C. to about 4° C.

60. The method of any one of claims 55-59, wherein upon contact of the coated organelle complexes population with the population of host cells, the coated organelle complexes have superior incorporation capability into host cells as compared to organelle complexes not comprising the one or more lipid-polymer conjugates, optionally said one or more lipid-polymer conjugates comprise a targeting agent configured to bind a ligand on the surface of said host cells.

61. A population of host cells comprising coated organelle complexes generated according to the method of any one of claims 55-60.

62. A composition comprising: (i) the coated organelle complexes population of any one of claim 1-39 or 54, or (ii) the population of host cells comprising coated organelle complexes of claim 61.

63. A pharmaceutical composition, comprising:
(i) the coated organelle complexes population of any one of claim 1-39 or 54, or (ii) the population of host cells comprising coated organelle complexes of claim 61; and
one or more pharmaceutically acceptable carriers,
optionally further comprising one or more secondary agents.

64. A method of treating or preventing a disease or disorder in a subject, the method comprising:
contacting cells of a subject in need thereof with an effective amount of:
(i) the coated organelle complexes population of any one of claim 1-39 or 54;
(ii) the population of host cells comprising coated organelle complexes of claim 61;
(iii) the composition of claim 62; and/or
(iv) the pharmaceutical composition of claim 63,
thereby treating or preventing the disease or disorder in the subject.

65. The method of claim 64, wherein the contacting is performed ex vivo, in vitro, or in vivo.

66. The method of any one of claims 64-65, wherein the effective amount comprises at least about 1 μg to about 1 mg of the coated organelle complexes population.

67. The method of any one of claims 64-66, wherein the subject is a mammal.

68. The method of any one of claims 64-67, wherein at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the coated organelle complexes population is incorporated into target cell(s) and/or target tissue(s) of the subject.

69. The method of any one of claims 64-68, wherein less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the coated organelle complexes population are incorporated into non-target cell(s) and/or non-target tissue(s) of the subject.

70. The method of any one of claims 64-69, wherein the disease or disorder is selected from the group consisting of diabetes (Type I and Type II), metabolic disease, ocular disorders associated with mitochondrial dysfunction, hearing loss, mitochondrial toxicity associated with therapeutic agents, mitochondrial dysfunction associated with Space travel, cardiotoxicity associated with chemotherapy or other therapeutic agents, a mitochondrial dysfunction disorder, and migraine.

71. The method of any one of claims 64-70, wherein the disease or disorder is selected from the group consisting of mitochondrial myopathy, diabetes and deafness (DAD) syndrome, Barth Syndrome, Leber's hereditary optic neuropathy (LHON), Leigh syndrome, NARP (neuropathy, ataxia, retinitis pigmentosa and ptosis syndrome), myoneurogenic gastrointestinal encephalopathy (MNGIE), MELAS (mitochondrial encephalopathy, lactic acidosis, and stroke-like episodes) syndrome, myoclonic epilepsy with ragged red fibers (MERRF) syndrome, Kearns-Sayre syndrome, and mitochondrial DNA depletion syndrome.

72. The method of any one of claims 64-71, wherein the disease or disorder is an ischemia-related disease or disorder, a genetic disorder, an aging disease or disorder, a neurodegenerative condition, a cardiovascular condition, a cancer, an autoimmune disease, an inflammatory disease, a fibrotic disorder, or any combination thereof.

73. The method of any one of claims 64-72, wherein the ischemia-related disease or disorder is selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, critical limb ischemia and reperfusion injury of a transplanted organ.

74. The method of any one of claims 64-73, wherein the neurodegenerative condition is selected from the group consisting of dementia, Friedrich's ataxia, amyotrophic lateral sclerosis, mitochondrial encephalopathy, lactic acidosis, and stroke-like episodes (MELAS), myoclonic epilepsy with ragged red fibers (MERFF), epilepsy, Parkinson's disease, Alzheimer's disease, or Huntington's Disease. Exemplary neuropsychiatric disorders include bipolar disorder, schizophrenia, depression, addiction disorders, anxiety disorders, attention deficit disorders, personality disorders, autism, and Asperger's discase.

75. The method of any one of claims 64-74, wherein the cardiovascular condition is selected from the group consisting of coronary heart disease, myocardial infarction, atherosclerosis, high blood pressure, cardiac arrest, cerebrovascular disease, peripheral arterial disease, rheumatic heart disease, congenital heart disease, congestive heart failure, arrhythmia, stroke, deep vein thrombosis, and pulmonary embolism.

76. The method of any one of claims 64-75, wherein the disease or disorder is acute respiratory distress syndrome (ARDS) or pre-eclampsia or intrauterine growth restriction (IUGR) or fetal growth restriction (FGR).

77. The method of any one of claims 64-76, wherein the disease or disorder is associated with expression of a tumor antigen, and wherein the disease associated with expression of a tumor antigen is selected from the group consisting of a proliferative disease, a precancerous condition, a cancer, and a non-cancer related indication associated with expression of the tumor antigen.

78. The method of any one of claims 64-77, wherein the cancer is selected from the group consisting of colon cancer, rectal cancer, renal-cell carcinoma, liver cancer, small cell or non-small cell carcinoma of the lung, mesothelioma, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers, combinations of said cancers, and metastatic lesions of said cancers.

79. The method of any one of claims 64-78, wherein the cancer is a hematologic cancer chosen from one or more of chronic lymphocytic leukemia (CLL), acute leukemias, acute lymphoid leukemia (ALL), B-cell acute lymphoid leukemia (B-ALL), T cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

80. The method of any one of claims 64-79, wherein the contacting comprises systemic administration, intrathecal administration, intracranial injection, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, inhalation, intrapulmonary administration, and intra-ocular administration, or any combination thereof, optionally the systemic administration is intravenous, intramuscular, intraperitoneal, or intraarticular.
